(19) United States
(12) Patent Application Publication
(10) Pub. No.: US 2013/0225633 A1
(43) Pub. Date: Aug. 29, 2013

(54) PHENYL HETEROCYCLOALKYL GLUCOCORTICOID RECEPTOR MODULATORS

(71) Applicant: Corcept Therapeutics, Inc., (US)

(72) Inventors: Hazel Hunt, Storrington (GB); Tony Johnson, Harlow (GB); Nicholas C. Ray, Harlow (GB)

(73) Assignee: Corcept Therapeutics, Inc., Menlo Park, CA (US)

(21) Appl. No.: 13/776,980

(22) Filed: Feb. 26, 2013

Related U.S. Application Data
(60) Provisional application No. 61/603,491, filed on Feb. 27, 2012.
(60) Provisional application No. 61/691,557, filed on Aug. 21, 2012.

(51) Int. Cl. C07D 471/04

(52) U.S. Cl. CPC C07D 471/04
USPC 514/293; 546/82

(57) ABSTRACT

The present invention provides phenyl-heterocycloalkyl fused azadecalin compounds and methods of using the compounds as glucocorticoid receptor modulators.

### CROSS-REFERENCES TO RELATED APPLICATIONS

This application claims priority to U.S. Provisional Application Nos. 61/603,491, filed Feb. 27, 2012, and 61/691,557, filed Aug. 21, 2012, which are incorporated in their entirety herein for all purposes.

### BACKGROUND OF THE INVENTION

In most species, including man, the physiological glucocorticoid is cortisol (hydrocortisone). Glucocorticoids are secreted in response to ACTH (corticotropin), which shows both circadian rhythm variation and elevations in response to stress and food. Cortisol levels are responsive within minutes to many physical and psychological stresses, including trauma, surgery, exercise, anxiety and depression. Cortisol is a steroid and acts by binding to an intracellular, glucocorticoid receptor (GR). In man, glucocorticoid receptors are present in two forms: a ligand-binding GR-alpha of 777 amino acids; and, a GR-beta isoform which lacks the 50 carboxy terminal residues. Since these include the ligand binding domain, GR-beta is unable to bind ligand, is constitutively localized in the nucleus, and is transcriptionally inactive. The GR is also known as the GR-II receptor.

The biologic effects of cortisol, including those caused by hypercortisolemia, can be modulated at the GR level using receptor modulators, such as agonists, partial agonists and antagonists. Several different classes of agents are able to block the physiologic effects of GR-agonist binding. These antagonists include compositions which, by binding to GR, block the ability of an agonist to effectively bind to and/or activate the GR. One such known GR antagonist, mifepristone, has been found to be an effective anti-glucocorticoid agent in humans (Bertagna (1984) J. Clin. Endocrinol. Metab. 59:25). Mifepristone binds to the GR with high affinity, with a dissociation constant (K_{d}) of 10⁻⁹ M (Cadepond (1997) Annu. Rev. Med. 48:129). What is needed in the art are new compositions and methods for modulating GR receptors. Surprisingly, the present invention meets these and other needs.

### BRIEF SUMMARY OF THE INVENTION

In one embodiment, the present invention provides a compound of formula I:

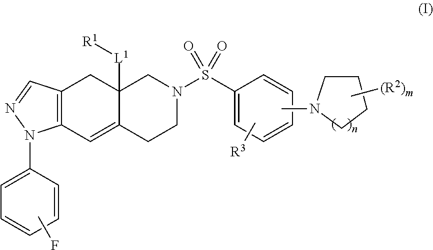

L¹ of formula I can be C₁₋₆ alkylene or —C(O)—. R¹ of formula I can be —OR^{1a}. Each R^{1a} of formula I can independently be C₁₋₆ alkyl, C₁₋₆ haloalkyl, C₃₋₈ cycloalkyl, or C₁₋₆ alkyl-C₃₋₈ cycloalkyl. R² of formula I can be hydrogen, halogen, C₁₋₆ alkyl, C₁₋₆ alkoxy, C₁₋₆ alkyl-C₁₋₆ alkoxy, C₁₋₆ haloalkyl, or C₁₋₆ haloalkoxy. R³ of formula I can be hydrogen, halogen, C₁₋₆ alkyl, or C₁₋₆ haloalkyl. Each of subscripts m and n of formula I can independently be 1 or 2. The salts and isomers of formula I are also included.

In another embodiment, the present invention provides a pharmaceutical composition including a pharmaceutically acceptable excipient and a compound of formula I.

In another embodiment, the present invention provides a method of modulating a glucocorticoid receptor, the method including contacting a glucocorticoid receptor with a compound of formula I, thereby modulating the glucocorticoid receptor.

In a fourth embodiment, the present invention provides a method of treating a disorder through antagonizing a glucocorticoid receptor, the method including administering to a subject in need of such treatment, a therapeutically effective amount of a compound of formula I, thereby treating the disorder.

### BRIEF DESCRIPTION OF THE DRAWINGS

FIGS. 1, 2, 3, 4 and 5 show various synthetic schemes for making the compounds of the present invention.

### DETAILED DESCRIPTION OF THE INVENTION

### I. General

The present invention provides compounds capable of modulating a glucocorticoid receptor (GR) and thereby providing beneficial therapeutic effects. The compounds include phenyl pyrrolidine fused azadecalins. The present invention also provides methods of treating diseases and disorders by modulating a GR receptor with the compounds of the present invention.

### II. Definitions

The abbreviations used herein have their conventional meaning within the chemical and biological arts.

Where substituent groups are specified by their conventional chemical formulae, written from left to right, they equally encompass the chemically identical substituents that would result from writing the structure from right to left, e.g., —CH₂O— is equivalent to —OCH₂—.

“Alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc.

“Alkylene” refers to a straight or branched, saturated, aliphatic radical having the number of carbon atoms indicated, and linking at least two other groups, i.e., a divalent hydrocarbon radical. The two moieties linked to the alkylene can be linked to the same atom or different atoms of the alkylene group. For instance, a straight chain alkylene can be the bivalent radical of —(CH₂)ₙ—, where n is 1, 2, 3, 4, 5 or 6. Representative alkylene groups include, but are not limited to, methylene, ethylene, propylene, isopropylene, butylene, isobutylene, sec-butylene, pentylene and hexylene.

“Alkoxy” refers to an alkyl group having an oxygen atom that connects the alkyl group to the point of attachment: alkyl-O—. As for alkyl group, alkoxy groups can have any suitable number of carbon atoms, such as C₁₋₆. Alkoxy groups include, for example, methoxy, ethoxy, propoxy, iso-propoxy, butoxy, 2-butoxy, iso-butoxy, sec-butoxy, tert-butoxy, pentoxy, hexoxy, etc.

“Alkyl-Alkoxy” refers to a radical having an alkyl component and an alkoxy component, where the alkyl component links the alkoxy component to the point of attachment. The alkyl component is as defined above, except that the alkyl component is at least divalent, an alkylene, to link to the alkoxy component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The alkoxy component is as defined above. Examples of the alkyl-alkoxy group include, but are not limited to, 2-ethoxy-ethyl and methoxymethyl.

“Halogen” refers to fluorine, chlorine, bromine and iodine.

“Haloalkyl” refers to alkyl, as defined above, where some or all of the hydrogen atoms are replaced with halogen atoms. As for alkyl groups, haloalkyl groups can have any suitable number of carbon atoms, such as C₁₋₆. For example, haloalkyl includes trifluoromethyl, fluoromethyl, etc. In some instances, the term “perfluoro” can be used to define a compound or radical where all the hydrogens are replaced with fluorine. For example, perfluoromethyl refers to 1,1,1-trifluoromethyl.

“Haloalkoxy” refers to an alkoxy group where some or all of the hydrogen atoms are substituted with halogen atoms. As for an alkyl group, haloalkoxy groups can have any suitable number of carbon atoms, such as C₁₋₆. The alkoxy groups can be substituted with 1, 2, 3, or more halogens. When all the hydrogens are replaced with a halogen, for example by fluorine, the compounds are per-substituted, for example, perfluorinated. Haloalkoxy includes, but is not limited to, trifluoromethoxy, 2,2,2,-trifluoroethoxy, perfluoroethoxy, etc.

“Cycloalkyl” refers to a saturated or partially unsaturated, monocyclic, fused bicyclic or bridged polycyclic ring assembly containing from 3 to 12 ring atoms, or the number of atoms indicated. Cycloalkyl can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane, [2.2.2]bicyclooctane, decahydronaphthalene and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene. When cycloalkyl is a saturated monocyclic C₃₋₈ cycloalkyl, exemplary groups include, but are not limited to cyclopropyl, cyclobutyl, cyclopentyl, cyclohexyl, cycloheptyl and cyclooctyl. When cycloalkyl is a saturated monocyclic C₃₋₆ cycloalkyl, exemplary groups include, but are not limited to cyclopropyl, cyclobutyl, cyclopentyl, and cyclohexyl.

“Alkyl-cycloalkyl” refers to a radical having an alkyl component and a cycloalkyl component, where the alkyl component links the cycloalkyl component to the point of attachment. The alkyl component is as defined above, except that the alkyl component is at least divalent, an alkylene, to link to the cycloalkyl component and to the point of attachment. In some instances, the alkyl component can be absent. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. The cycloalkyl component is as defined within. Exemplary alkyl-cycloalkyl groups include, but are not limited to, methyl-cyclopropyl, methyl-cyclobutyl, methyl-cyclopentyl and methyl-cyclohexyl.

“Heterocycloalkyl” refers to a saturated ring system having from 3 to 12 ring members and from 1 to 3 heteroatoms of N, O and S. Additional heteroatoms can also be useful, including, but not limited to, B, Al, Si and P. The heteroatoms can also be oxidized, such as, but not limited to, —S(O)— and —S(O)₂—. Heterocycloalkyl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heterocycloalkyl groups, such as 1, 2, 3, or 1 to 2, 1 to 3, or 2 to 3. The heterocycloalkyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane, oxazolidine, isoxazollidine, thiazolidine, isothiazolidine, dioxolane, dithiolane, morpholine, thiomorpholine, dioxane, or dithiane. The heterocycloalkyl groups can also be fused to aromatic or non-aromatic ring systems to form members including, but not limited to, indoline.

When heterocycloalkyl includes 3 to 8 ring members and 1 to 3 heteroatoms, representative members include, but are not limited to, pyrrolidine, piperidine, tetrahydrofuran, oxane, tetrahydrothiophene, thiane, pyrazolidine, imidazolidine, piperazine, oxazolidine, isoxzoalidine, thiazolidine, isothiazolidine, morpholine, thiomorpholine, dioxane and dithiane. Heterocycloalkyl can also form a ring having 5 to 6 ring members and 1 to 2 heteroatoms, with representative members including, but not limited to, pyrrolidine, piperidine, tetrahydrofuran, tetrahydrothiophene, pyrazolidine, imidazolidine, piperazine, oxazolidine, isoxazolidine, thiazolidine, isothiazolidine, and morpholine.

“Salt” refers to acid or base salts of the compounds used in the methods of the present invention. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

“Hydrate” refers to a compound that is complexed to at least one water molecule. The compounds of the present invention can be complexed with from 1 to 10 water molecules.

“Isomers” refers to compounds with the same chemical formula but which are structurally distinguishable.

“Tautomer” refers to one of two or more structural isomers which exist in equilibrium and which are readily converted from one form to another.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors and colors, and the like. One of skill in the art will recognize that other pharmaceutical excipients are useful in the present invention.

“Modulating a glucocorticoid receptor” refers to methods for adjusting the response of a glucocorticoid receptor towards glucocorticoids, glucocorticoid antagonists, agonists, and partial agonists. The methods include contacting a glucocorticoid receptor with an effective amount of either an antagonist, an agonist, or a partial agonist and detecting a change in GR activity.

“Glucocorticoid receptor” (“GR”) refers to a family of intracellular receptors which specifically bind to cortisol and/or cortisol analogs (e.g. dexamethasone). The glucocorticoid receptor is also referred to as the cortisol receptor. The term includes isoforms of GR, recombinant GR and mutated GR.

“Glucocorticoid receptor antagonist” refers to any composition or compound which partially or completely inhibits (antagonizes) the binding of a glucocorticoid receptor (GR) agonist, such as cortisol, or cortisol analogs, synthetic or natural, to a GR. A “specific glucocorticoid receptor antagonist” refers to any composition or compound which inhibits any biological response associated with the binding of a GR to an agonist. By “specific,” we intend the drug to preferentially bind to the GR rather than other nuclear receptors, such as mineralocorticoid receptor (MR) or progesterone receptor (PR).

“GR modulator” refers to compounds that agonize and/or antagonize the glucocorticoid receptor and are defined as compounds of Formula I below.

“Treat”, “treating” and “treatment” refer to any indicia of success in the treatment or amelioration of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation.

“Patient” or “subject in need thereof” refers to a living organism suffering from or prone to a condition that can be treated by administration of a pharmaceutical composition as provided herein. Non-limiting examples include humans, other mammals and other non-mammalian animals.

“Disorder” or “condition” refer to a state of being or health status of a patient or subject capable of being treated with the glucocorticoid receptor modulators of the present invention. Examples of disorders or conditions include, but are not limited to, obesity, hypertension, depression, anxiety, and Cushing's Syndrome.

“Antagonizing” refers to blocking the binding of an agonist at a receptor molecule or to inhibiting the signal produced by a receptor-agonist. A receptor antagonist blocks or dampens agonist-mediated responses.

“Therapeutically effective amount” refers to an amount of a conjugated functional agent or of a pharmaceutical composition useful for treating or ameliorating an identified disease or condition, or for exhibiting a detectable therapeutic or inhibitory effect. The effect can be detected by any assay method known in the art.

Description of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, or physiological conditions.

### III. Compounds

The present invention provides many fused azadecalin compounds. In some embodiments, the present invention provides compounds having the structure of formula I:

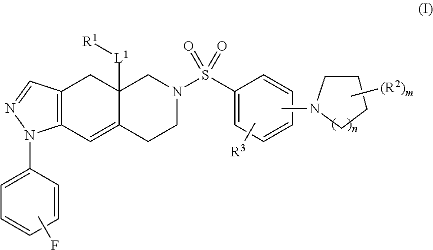

L¹ of formula I can be C₁₋₆ alkylene or —C(O)—. R¹ of formula I can be —OR^{1a}. Each R^{1a} of formula I can independently be C₁₋₆ alkyl, C₁₋₆ haloalkyl, C₃₋₈ cycloalkyl, or C₁₋₆ alkyl-C₃₋₈ cycloalkyl. R² of formula I can be hydrogen, halogen, C₁₋₆ alkyl, C₁₋₆ alkoxy, C₁₋₆ alkyl-C₁₋₆ alkoxy, C₁₋₆ haloalkyl, or C₁₋₆ haloalkoxy. R³ of formula I can be hydrogen, halogen, C₁₋₆ alkyl, or C₁₋₆ haloalkyl. Each of subscripts m and n of formula I can independently be 1 or 2. The salts and isomers of formula I are also included.

In other embodiments, the compounds have the following structure:

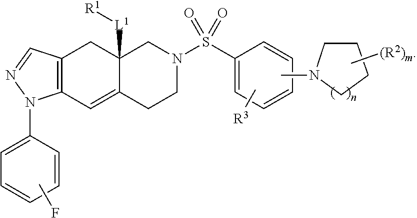

The L¹-R¹ group can be any suitable group. In some embodiments, the group L¹-R¹ can be —CH₂OR^{1a} or —C(O)OR^{1a}. In other embodiments, R^{1a} can be C₁₋₆ alkyl, C₁₋₆ haloalkyl, or C₁₋₆ alkyl-C₃₋₈ cycloalkyl. In some other embodiments, the group L¹-R¹ can be methoxymethyl, ethoxymethyl, isopropoxymethyl, (fluoromethoxy)methyl, (difluoromethoxy)methyl, (trifluoromethoxy)methyl, (cyclopropylmethoxy)methyl, (cyclobutylmethoxy)methyl, methyl carboxylate, ethyl carboxylate, isopropyl carboxylate, fluoromethyl carboxylate, cyclopropyl carboxylate, cyclobutyl carboxylate, cyclopropylmethyl carboxylate, or cyclobutylmethyl carboxylate. In still other embodiments, the group L¹-R¹ can be methoxymethyl, ethoxymethyl, (cyclopropylmethoxy)methyl, methyl carboxylate, ethyl carboxylate or fluoromethyl carboxylate.

The R² group can be any suitable group. In some embodiments, R² can be hydrogen, halogen, or C₁₋₆ alkoxy, while subscript m can be 1. In other embodiments, R² can be H or F.

In some embodiments, R^{1a} can be C₁₋₆ alkyl, C₁₋₆ haloalkyl, or C₁₋₆alkylC₃₋₈ cycloalkyl, R² can be hydrogen, halogen, or C₁₋₆ alkoxy, R³ can be H, while each of subscripts m and n can be 1.

In some embodiments, R³ can be H, while subscripts m and n are each 1. In other embodiments, the compounds of formula I have the structure:

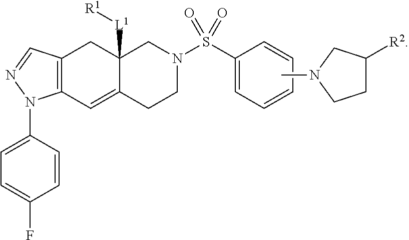

In some other embodiments, the compounds of formula I have the structure:

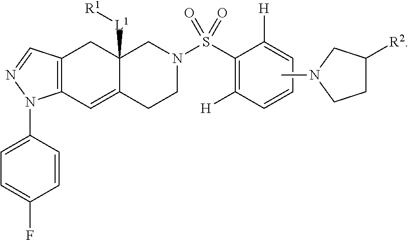

In other embodiments, the group L¹-R¹ can be methoxymethyl, ethoxymethyl, (cyclopropylmethoxy)methyl, methyl carboxylate, ethyl carboxylate or fluoromethyl carboxylate, R² can be H or F, R³ can be H, while each of subscripts m and n can be 1.

In some other embodiments, the compounds of formula I can have the structure:

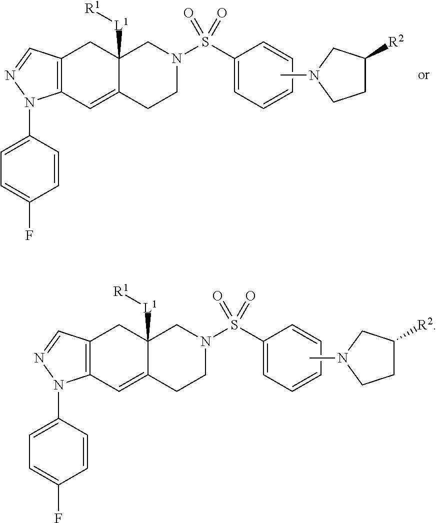

In some embodiments, the compounds of formula I can have the structure:

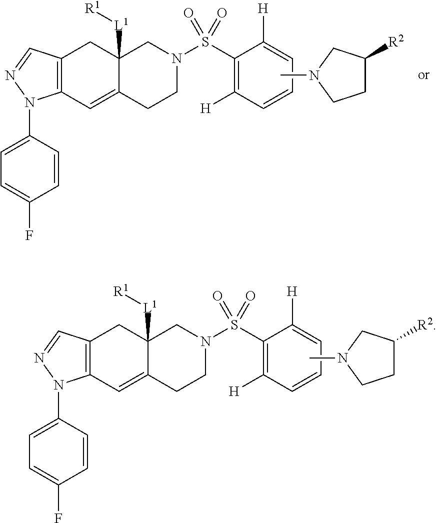

In other embodiments, the compounds of formula I can be:
(R)-4a-Cyclopropylmethoxymethyl-1-(4-fluorophenyl)-6-[3-((R)-3-fluoropyrrolidin-1-yl)-benzenesulfonyl]-4,4a,5,6,7,8-hexahydro-1H-1,2,6-triaza-cyclopenta[b]naphthalene;
(R)-4a-Cyclopropylmethoxymethyl-1-(4-fluorophenyl)-6-[3-((S)-3-fluoropyrrolidin-1-yl)-phenyl-3-sulfonyl]-4,4a,5,6,7,8-hexahydro-1H-1,2,6-triaza-cyclopenta[b]naphthalene;
(R)-4a-Cyclopropylmethoxymethyl-1-(4-fluorophenyl)-6-[3-(pyrrolidin-1-yl)-benzenesulfonyl]-4,4a,5,6,7,8-hexahydro-1H-1,2,6-triaza-cyclopenta[b]naphthalene;
(R)-1-(4-Fluorophenyl)-6-[3-((R)-3-fluoropyrrolidin-1-yl)-benzenesulfonyl]-4a-methoxymethyl-4,4a,5,6,7,8-hexahydro-1H-1,2,6-triaza-cyclopenta[b]naphthalene;
(R)-1-(4-Fluorophenyl)-6-[3-((S)-3-fluoropyrrolidin-1-yl)-benzenesulfonyl]-4a-methoxymethyl-4,4a,5,6,7,8-hexahydro-1H-1,2,6-triaza-cyclopenta[b]naphthalene;
(R)-1-(4-Fluoro-phenyl)-4a-methoxymethyl-6-[3-(-pyrrolidin-1-yl)-benzenesulfonyl)-4,4a,5,6,7,8-hexahydro-1H-1,2,6-triaza-cyclopenta[b]naphthalene;
(R)-4a-Cyclopropylmethoxymethyl-1-(4-fluoro-phenyl)-6-[4-((R)-3-fluoro-pyrrolidin-1-yl)-benzenesulfonyl]-4,4a,5,6,7,8-hexahydro-1H-1,2,6-triaza-cyclopenta[b]naphthalene;
(R)-4a-Cyclopropylmethoxymethyl-1-(4-fluoro-phenyl)-6-[4-(pyrrolidin-1-yl)-benzenesulfonyl]-4,4a,5,6,7,8-hexahydro-1H-1,2,6-triaza-cyclopenta[b]naphthalene;
(R)-1-(4-Fluoro-phenyl)-6-[4-((R)-3-fluoro-pyrrolidin-1-yl)-benzenesulfonyl]-4a-methoxymethyl-4,4a,5,6,7,8-hexahydro-1H-1,2,6-triaza-cyclopenta[b]naphthalene;
(R)-1-(4-Fluoro-phenyl)-6-[4-(pyrrolidin-1-yl)-benzenesulfonyl]-4a-methoxymethyl-4,4a,5,6,7,8-hexahydro-1H-1,2,6-triaza-cyclopenta[b]naphthalene;
(R)-1-(4-Fluoro-phenyl)-6-[3-((R)-3-fluoro-pyrrolidin-1-yl)-benzenesulfonyl]-1,4,5,6,7,8-hexahydro-1,2,6-triaza-cyclopenta[b]naphthalene-4a-carboxylic acid ethyl ester;
(R)-1-(4-Fluoro-phenyl)-6-[3-((R)-3-fluoro-pyrrolidin-1-yl)-benzenesulfonyl]-1,4,5,6,7,8-hexahydro-1,2,6-triaza-cyclopenta[b]naphthalene-4a-carboxylic acid methyl ester;
(R)-1-(4-Fluoro-phenyl)-6-[3-((R)-3-fluoro-pyrrolidin-1-yl)-benzenesulfonyl]-1,4,5,6,7,8-hexahydro-1,2,6-triaza-cyclopenta[b]naphthalene-4a-carboxylic acid fluoromethyl ester;
(R)-1-(4-Fluoro-phenyl)-6-[3-(pyrrolidin-1-yl)-benzenesulfonyl]-1,4,5,6,7,8-hexahydro-1,2,6-triaza-cyclopenta[b]naphthalene-4a-carboxylic acid ethyl ester;
(R)-1-(4-Fluoro-phenyl)-6-[3-(pyrrolidin-1-yl)-benzenesulfonyl]-1,4,5,6,7,8-hexahydro-1,2,6-triaza-cyclopenta[b]naphthalene-4a-carboxylic acid fluoromethyl ester,
(R)-1-(4-Fluoro-phenyl)-6-[4-(3,3-difluoropyrrolidin-1-yl)-benzenesulfonyl]-4a-methoxymethyl-4,4a,5,6,7,8-hexahydro-1H-1,2,6-triaza-cyclopenta[b]naphthalene,
(R)-1-(4-Fluoro-phenyl)-6-[4-((R)-2-methylpyrrolidin-1-yl)-benzenesulfonyl]-4a-methoxymethyl-4,4a,5,6,7,8-hexahydro-1H-1,2,6-triaza-cyclopenta[b]naphthalene,
(R)-1-(4-Fluoro-phenyl)-6-[4-((S)-2-(methoxymethyl)pyrrolidin-1-yl)-benzenesulfonyl]-4a-methoxymethyl-4,4a,5,6,7,8-hexahydro-1H-1,2,6-triaza-cyclopenta[b]naphthalene,
(R)-1-(4-Fluoro-phenyl)-6-[4-((S)-3-methoxypyrrolidin-1-yl)-benzenesulfonyl]-4a-methoxymethyl-4,4a,5,6,7,8-hexahydro-1H-1,2,6-triaza-cyclopenta[b]naphthalene,
(R)-1-(4-Fluoro-phenyl)-6-[4-(piperidin-1-yl)-benzenesulfonyl]-4a-methoxymethyl-4,4a,5,6,7,8-hexahydro-1H-1,2,6-triaza-cyclopenta[b]naphthalene, or
(R)-1-(4-Fluoro-phenyl)-6-[4-(3,3-dimethylpyrrolidin-1-yl)-benzenesulfonyl]-4a-methoxymethyl-4,4a,5,6,7,8-hexahydro-1H-1,2,6-triaza-cyclopenta[b]naphthalene.

The compounds of the present invention can exist as salts. The present invention includes such salts. Examples of applicable salt forms include hydrochlorides, hydrobromides, sulfates, methanesulfonates, nitrates, maleates, acetates, citrates, fumarates, tartrates (eg (+)-tartrates, (−)-tartrates or mixtures thereof including racemic mixtures), succinates, benzoates and salts with amino acids such as glutamic acid. These salts may be prepared by methods known to those skilled in art. When compounds of the present invention contain relatively basic functionalities, acid addition salts can be obtained by contacting the neutral form of such compounds with a sufficient amount of the desired acid, either neat or in a suitable inert solvent. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain acidic functionalities that allow the compounds to be converted into base addition salts. Additional information on suitable pharmaceutically acceptable salts can be found in Remington's Pharmaceutical Sciences, 17th ed., Mack Publishing Company, Easton, Pa., 1985, which is incorporated herein by reference.

The neutral forms of the compounds are preferably regenerated by contacting the salt with a base or acid and isolating the parent compound in the conventional manner. The parent form of the compound differs from the various salt forms in certain physical properties, such as solubility in polar solvents.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques.

Isomers include compounds having the same number and kind of atoms, and hence the same molecular weight, but differing in respect to the structural arrangement or configuration of the atoms.

It will be apparent to one skilled in the art that certain compounds of this invention may exist in tautomeric forms, all such tautomeric forms of the compounds being within the scope of the invention. Tautomer refers to one of two or more structural isomers which exist in equilibrium and which are readily converted from one isomeric form to another.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, the compounds of the present invention may also contain unnatural proportions of atomic isotopes at one or more of the atoms that constitute such compounds. For example, the compounds of the present invention may be radiolabeled with radioactive isotopes, such as for example deuterium (²H), tritium (³H), iodine-125 (¹²⁵I), carbon-13 (¹³C), or carbon-14 (¹⁴C). All isotopic variations of the compounds of the present invention, whether radioactive or not, are encompassed within the scope of the present invention.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

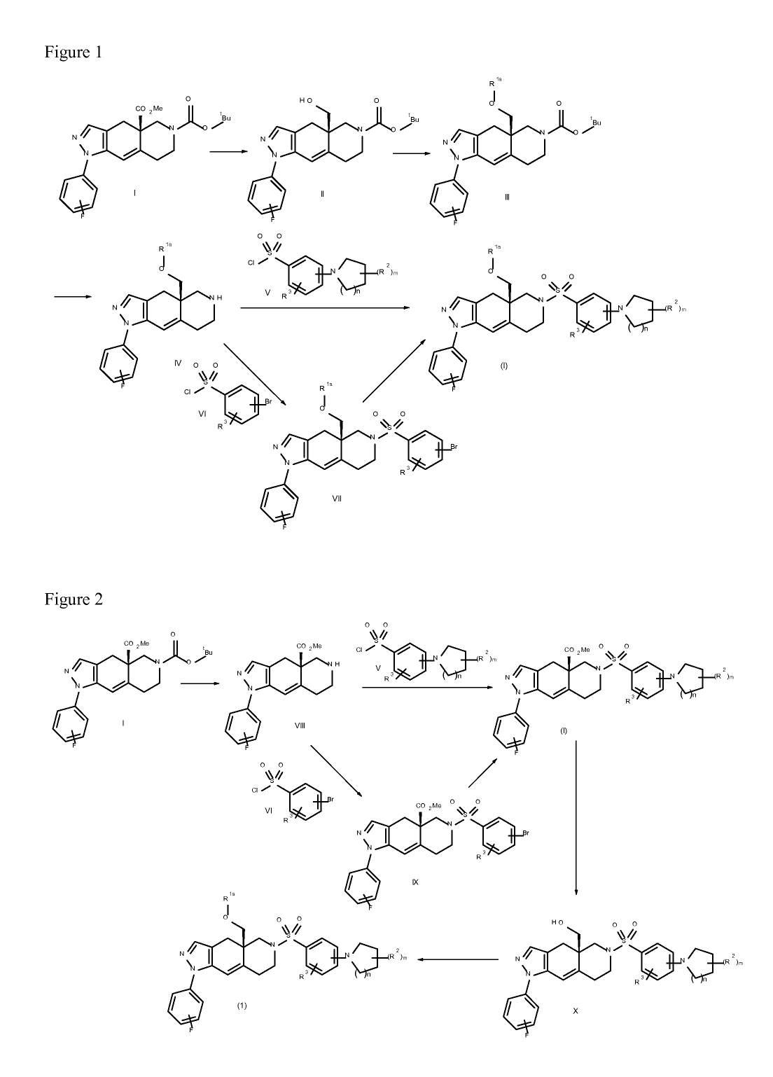

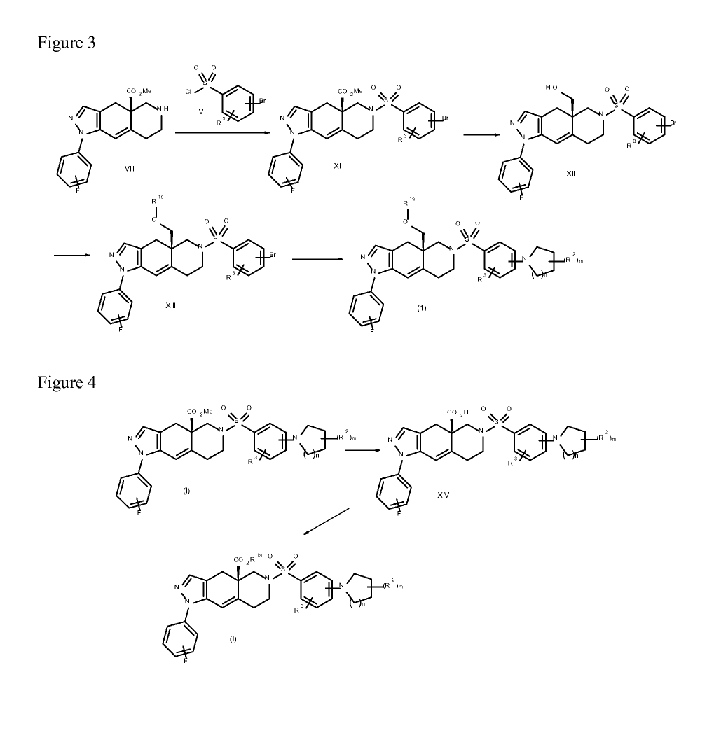

The compounds of the invention can be synthesized by a variety of methods known to one of skill in the art (see Comprehensive Organic Transformations Richard C. Larock, 1989) or by an appropriate combination of generally well known synthetic methods. Techniques useful in synthesizing the compounds of the invention are both readily apparent and accessible to those of skill in the relevant art. The discussion below is offered to illustrate certain of the diverse methods available for use in assembling the compounds of the invention. However, the discussion is not intended to define the scope of reactions or reaction sequences that are useful in preparing the compounds of the present invention. One of skill in the art will appreciate that other methods of making the compounds are useful in the present invention. Although some compounds in FIG. 1, FIG. 2, and Table 1 may indicate relative stereochemistry, the compounds may exist as a racemic mixture or as either enantiomer.

Compounds of the present invention in which R¹-L¹ represents CH₂OR^{1a} can be prepared as shown in FIG. 1. Starting materials can be obtained from commercial sources, by employing known synthetic methods, and by employing methods described in U.S. Pat. No. 7,928,237, incorporated herein by reference. Esters I are converted to alcohols II by treatment with a reducing agent such as DIBAL-H, LiAlH₄ or RED-AL, preferably DIBAL-H in an inert solvent such as dichloromethane, tetrahydrofuran, benzene or toluene, preferably dichloromethane. Alcohols II are converted into ether derivatives III by treatment with a base (e.g. sodium hydride) in an aprotic solvent such as tetrahydrofuran, N,N-dimethylformamide, preferably tetrahydrofuran, followed by addition of an alkyl, haloalkyl, cycloalkyl or cycloalkylalkyl halide or a methanesulfonate. Alternatively, the alkylation can be achieved by using phase transfer conditions, such as sodium hydroxide, tetrabutylammoniumhydrogensulfate, tetrabutylammonium iodide and an alkyl (or haloalkyl, cycloalkyl or cycloalkylalkyl) halide in aqueous tetrahydrofuran. The tert-butoxycarbonyl protecting group is removed from III by treatment with an acid, such as HCl, HBr, trifluoroacetic acid, p-toluenesulfonic acid or methanesulfonic acid, preferably HCl or trifluoroacetic acid, optionally in a solvent such as dioxane, ethanol or tetrahydrofuran, preferably dioxane, either under anhydrous or aqueous conditions. Amines IV are converted to the compounds of formula (I) by treatment with an appropriate amino-substituted benzenesulfonyl halide, such as the benzenesulfonyl chloride V, in an inert solvent such as dichloromethane, toluene or tetrahydrofuran, preferably dichloromethane, in the presence of a base such as N,N-diisopropylethylamine or triethylamine. It can be convenient to carry out the sulfonylation reaction in situ, without isolation of the amine IV. Compounds of formula (I) can also be prepared from amines of formula IV in a two-step sequence beginning with reaction of amines IV with a bromo (or chloro)-substituted benzene-sulfonylchloride, such as bromobenzene-sulfonyl chloride VI, to afford a halo-substituted benzenesulfonamide derivative exemplified by VII. Treatment of VII with an amine in an inert solvent, such as tetrahydrofuran, toluene or N,N-dimethylformamide, optionally in the presence of a palladium catalyst (e.g. BINAP/Pd₂(dba)₃) and a base (e.g. sodium or potassium tert-butoxide), optionally under microwave conditions, affords compounds of formula (I).

Compounds of the present invention can also be prepared as shown in FIG. 2. The tert-butoxycarbonyl protecting group is removed from the protected amine I by treatment with an acid, such as HCl, HBr, trifluoroacetic acid, p-toluenesulfonic acid or methanesulfonic acid, preferably HCl or trifluoroacetic acid, optionally in a solvent such as dioxane, ethanol or tetrahydrofuran, preferably dioxane, either under anhydrous or aqueous conditions to afford amines VIII. Amines VIII are converted to the benzenesulfonamides of formula (I) by treatment with an amino-substituted benzenesulfonyl halide, such as the benzenesulfonylchloride V, in an inert solvent such as dichloromethane, toluene or tetrahydrofuran, preferably dichloromethane, in the presence of a base such as N,N-diisopropylethylamine or triethylamine. Alternatively, amines VIII can be converted into benzenesulfonamides of formula (I) in a two-step process, involving sulfonylation with an appropriate halo substituted benzenesulfonyl chloride of formula VI to provide a sulfonamide of formula IX, and subsequent conversion of the halo substituent to the required amino substituent as described for FIG. 1. It can be convenient to carry out the sulfonylation in situ, without isolation of the amine VIII. Reduction of the methyl ester group in esters of formula (I) by treatment with a reducing agent such as DIBAL-H, LiAlH₄ or RED-AL, preferably DIBAL-H in an inert solvent such as dichloromethane, tetrahydrofuran, benzene or toluene, preferably dichloromethane, affords alcohols X. Alcohols X can be converted into compounds of formula (I) by treatment with a base (e.g. sodium hydride) in an aprotic solvent such as acetonitrile, dimethylsulfoxide, tetrahydrofuran or N,N-dimethylformamide, preferably tetrahydrofuran, followed by addition of an alkyl, haloalkyl, cycloalkyl or cycloalkylalkyl halide or methanesulfonate. Alternatively, the alkylation can be achieved by using phase transfer conditions, such as sodium hydroxide, tetrabutylammoniumhydrogensulfate, tetrabutylammonium iodide and an alkyl (or haloalkyl, cycloalkyl or cycloalkylalkyl) halide in aqueous tetrahydrofuran.

Compounds of the invention can also be prepared as shown in FIG. 3. Amines VIII can be converted to the bromobenzenesulfonamides XI by treatment with a bromo-substituted benzenesulfonyl halide, such as the bromo-benzenesulfonylchloride VI, in an inert solvent such as dichloromethane, toluene or tetrahydrofuran, preferably dichloromethane, in the presence of a base such as N,N-diisopropylethylamine or triethylamine. The methyl ester group in compounds of XI can be reduced by treatment with a reducing agent such as DIBAL-H, LiAlH₄ or RED-AL, preferably DIBAL-H, in an inert solvent such as dichloromethane, tetrahydrofuran, benzene or toluene, preferably dichloromethane, to provide alcohols XII. Alcohols XII can be converted into ethers XIII by treatment with a base (e.g. sodium hydride) in an aprotic solvent such as acetonitrile, dimethylsulfoxide, tetrahydrofuran or N,N-dimethylformamide, preferably tetrahydrofuran, followed by addition of an alkyl, haloalkyl, cycloalkyl or cycloalkylalkyl halide or methanesulfonate. Alternatively, the alkylation can be achieved by using phase transfer conditions, such as sodium hydroxide, tetrabutylammoniumhydrogensulfate, tetrabutylammonium iodide and an alkyl (or haloalkyl, cycloalkyl or cycloalkylalkyl) halide in aqueous tetrahydrofuran. Bromo compound XIII can be converted into compounds of formula (I) by treatment with an amine in an inert solvent, such as tetrahydrofuran, toluene or N,N-dimethylformamide, optionally in the presence of a palladium catalyst (e.g. BINAP/Pd₂(dba)₃) and a base (e.g. sodium or potassium tert-butoxide), optionally under microwave conditions.

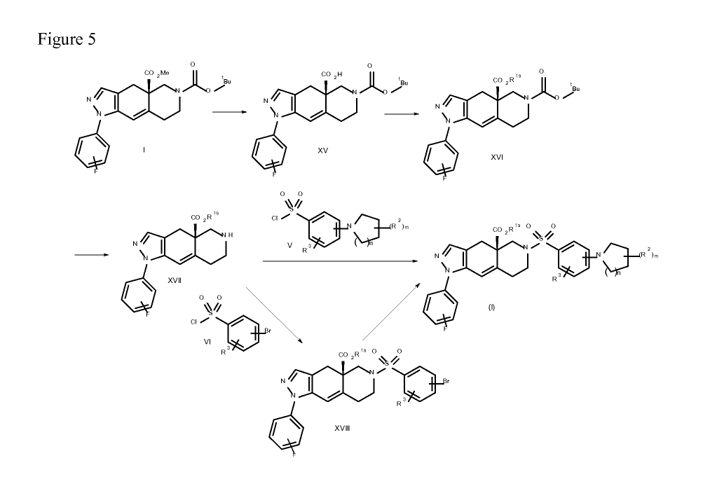

Compounds of the present invention in which R¹-L¹ represents CO₂R^{1a} can be prepared as shown in FIG. 4. Hydrolysis of methyl esters of formula (I) by treatment with a base, such as lithium hydroxide, in aqueous tetrahydrofuran affords acids XIV which can be converted into esters by conversion to the acid chloride, e.g., by reaction with oxalyl chloride in dichloromethane, followed by treatment with the requisite alcohol in dichloromethane in the presence of triethylamine. Alternatively, acids XIV can be alkylated with the appropriate alkyl (or haloalkyl, cycloalkyl or cycloalkylalkyl) halide under phase transfer conditions (tetrabutylammonium iodide, aqueous sodium hydroxide, tetrahydrofuran) or in N,N-dimethylformamide with cesium carbonate as base to form esters of formula (I).

Compounds of the present invention can also be prepared as shown in FIG. 5. Acids XV, prepared by hydrolysis of methyl esters I, can be converted to esters XVI as described for the preparation of esters of formula (I) in FIG. 4. Conversion of esters XVI to compounds of formula (I) can be effected as described for the preparation of compounds of formula (I) in FIG. 1, beginning with removal of the tert-butoxycarbonyl protecting group from XVI. The resulting amine XVII can then be converted to a compound of formula (I) by reaction with an amino-substituted benzenesulfonyl chloride V. Alternatively, the ester-amine XVII can be reacted with a halo-substituted benzenesulfonyl chloride VI to afford a halo-substituted benzenesulfonamide derivative XVIII, which can then be treated with an amine to afford a compound of formula (I).

### IV. Pharmaceutical Compositions

In some embodiments, the present invention provides a pharmaceutical composition including a pharmaceutically acceptable excipient and the compound of formula I.

The compounds of the present invention can be prepared and administered in a wide variety of oral, parenteral and topical dosage forms. Oral preparations include tablets, pills, powder, dragees, capsules, liquids, lozenges, gels, syrups, slurries, suspensions, etc., suitable for ingestion by the patient. The compounds of the present invention can also be administered by injection, that is, intravenously, intramuscularly, intracutaneously, subcutaneously, intraduodenally, or intraperitoneally. Also, the compounds described herein can be administered by inhalation, for example, intranasally. Additionally, the compounds of the present invention can be administered transdermally. The GR modulators of this invention can also be administered by in intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995). Accordingly, the present invention also provides pharmaceutical compositions including a pharmaceutically acceptable carrier or excipient and either a compound of Formula (I), or a pharmaceutically acceptable salt of a compound of Formula (I).

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton Pa. (“Remington's”).

In powders, the carrier is a finely divided solid, which is in a mixture with the finely divided active component. In tablets, the active component is mixed with the carrier having the necessary binding properties in suitable proportions and compacted in the shape and size desired.

The powders and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

Suitable solid excipients are carbohydrate or protein fillers including, but are not limited to, sugars, including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; as well as proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, for example, push-fit capsules made of gelatin, as well as soft, sealed capsules made of gelatin and a coating such as glycerol or sorbitol. Push-fit capsules can contain GR modulator mixed with a filler or binders such as lactose or starches, lubricants such as talc or magnesium stearate, and, optionally, stabilizers. In soft capsules, the GR modulator compounds may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending a GR modulator in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The GR modulators of the invention can be delivered by transdermally, by a topical route, formulated as applicator sticks, solutions, suspensions, emulsions, gels, creams, ointments, pastes, jellies, paints, powders, and aerosols.

The GR modulators and compositions of the invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

The GR modulator pharmaceutical formulations of the invention can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use

In another embodiment, the GR modulator formulations of the invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the GR modulator into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg, more typically 1.0 mg to 1000 mg, most typically 10 mg to 500 mg, according to the particular application and the potency of the active component. The composition can, if desired, also contain other compatible therapeutic agents.

The dosage regimen also takes into consideration pharmacokinetics parameters well known in the art, i.e., the rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, GR modulator and disease or condition treated.

Single or multiple administrations of GR modulator formulations can be administered depending on the dosage and frequency as required and tolerated by the patient. The formulations should provide a sufficient quantity of active agent to effectively treat the disease state. Thus, in one embodiment, the pharmaceutical formulations for oral administration of GR modulator is in a daily amount of between about 0.5 to about 20 mg per kilogram of body weight per day. In an alternative embodiment, dosages are from about 1 mg to about 4 mg per kg of body weight per patient per day are used. Lower dosages can be used, particularly when the drug is administered to an anatomically secluded site, such as the cerebral spinal fluid (CSF) space, in contrast to administration orally, into the blood stream, into a body cavity or into a lumen of an organ. Substantially higher dosages can be used in topical administration. Actual methods for preparing parenterally administrable GR modulator formulations will be known or apparent to those skilled in the art and are described in more detail in such publications as Remington's, supra. See also Nieman, In “Receptor Mediated Antisteroid Action,” Agarwal, et al., eds., De Gruyter, New York (1987).

The compounds described herein can be used in combination with one another, with other active agents known to be useful in modulating a glucocorticoid receptor, or with adjunctive agents that may not be effective alone, but may contribute to the efficacy of the active agent.

In some embodiments, co-administration includes administering one active agent within 0.5, 1, 2, 4, 6, 8, 10, 12, 16, 20, or 24 hours of a second active agent. Co-administration includes administering two active agents simultaneously, approximately simultaneously (e.g., within about 1, 5, 10, 15, 20, or 30 minutes of each other), or sequentially in any order. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

After a pharmaceutical composition including a GR modulator of the invention has been formulated in an acceptable carrier, it can be placed in an appropriate container and labeled for treatment of an indicated condition. For administration of GR modulators, such labeling would include, e.g., instructions concerning the amount, frequency and method of administration.

The pharmaceutical compositions of the present invention can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use.

In another embodiment, the compositions of the present invention are useful for parenteral administration, such as intravenous (IV) administration or administration into a body cavity or lumen of an organ. The formulations for administration will commonly comprise a solution of the compositions of the present invention dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present invention in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the formulations of the compositions of the present invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

### V. Method of Treating Via Glucocorticoid Modulation

In some embodiments, the present invention provides a method of treating a disorder or condition through modulating a glucocorticoid receptor, the method including administering to a subject in need of such treatment, a therapeutically effective amount of a compound of formula I.

In some other embodiments, the present invention provides a method of treating a disorder or condition through antagonizing a glucocorticoid receptor, the method including administering to a subject in need of such treatment, an effective amount of the compound of formula I.

In another embodiment, the present invention provides methods of modulating glucocorticoid receptor activity using the techniques described herein. In an exemplary embodiment, the method includes contacting a GR with an effective amount of a compound of the present invention, such as the compound of formula I, and detecting a change in GR activity.

In an exemplary embodiment, the GR modulator is an antagonist of GR activity (also referred to herein as “a glucocorticoid receptor antagonist”). A glucocorticoid receptor antagonist, as used herein, refers to any composition or compound which partially or completely inhibits (antagonizes) the binding of a glucocorticoid receptor (GR) agonist (e.g. cortisol and synthetic or natural cortisol analog) to a GR thereby inhibiting any biological response associated with the binding of a GR to the agonist.

In a related embodiment, the GR modulator is a specific glucocorticoid receptor antagonist. As used herein, a specific glucocorticoid receptor antagonist refers to a composition or compound which inhibits any biological response associated with the binding of a GR to an agonist by preferentially binding to the GR rather than another nuclear receptor (NR). In some embodiments, the specific glucocorticoid receptor antagonist binds preferentially to GR rather than the mineralocorticoid receptor (MR) or progesterone receptor (PR). In an exemplary embodiment, the specific glucocorticoid receptor antagonist binds preferentially to GR rather than the mineralocorticoid receptor (MR). In another exemplary embodiment, the specific glucocorticoid receptor antagonist binds preferentially to GR rather than the progesterone receptor (PR).

In a related embodiment, the specific glucocorticoid receptor antagonist binds to the GR with an association constant (K_{d}) that is at least 10-fold less than the K_{d} for the NR. In another embodiment, the specific glucocorticoid receptor antagonist binds to the GR with an association constant (K_{d}) that is at least 100-fold less than the K_{d} for the NR. In another embodiment, the specific glucocorticoid receptor antagonist binds to the GR with an association constant (K_{d}) that is at least 1000-fold less than the K_{d} for the NR.

Examples of disorders or conditions suitable for use with present invention include, but are not limited to, obesity, diabetes, cardiovascular disease, hypertension, Syndrome X, depression, anxiety, glaucoma, human immunodeficiency virus (HIV) or acquired immunodeficiency syndrome (AIDS), neurodegeneration, Alzheimer's disease, Parkinson's disease, cognition enhancement, Cushing's Syndrome, Addison's Disease, osteoporosis, frailty, muscle frailty, inflammatory diseases, osteoarthritis, rheumatoid arthritis, asthma and rhinitis, adrenal function-related ailments, viral infection, immunodeficiency, immunomodulation, autoimmune diseases, allergies, wound healing, compulsive behavior, multi-drug resistance, addiction, psychosis, anorexia, cachexia, post-traumatic stress syndrome, post-surgical bone fracture, medical catabolism, major psychotic depression, mild cognitive impairment, psychosis, dementia, hyperglycemia, stress disorders, antipsychotic induced weight gain, delirium, cognitive impairment in depressed patients, cognitive deterioration in individuals with Down's syndrome, psychosis associated with interferon-alpha therapy, chronic pain, pain associated with gastroesophageal reflux disease, postpartum psychosis, postpartum depression, neurological disorders in premature infants, and migraine headaches. In some embodiments, the disorder or condition can be major psychotic depression, stress disorders or antipsychotic induced weight gain. In other embodiments, the disorder or condition can be Cushing's Syndrome.

### VI. Assays and Methods for Modulating Glucocorticoid Receptor Activity

The compounds of the present invention can be tested for their antiglucocorticoid properties. Methods of assaying compounds capable of modulating glucocorticoid receptor activity are presented herein. Typically, compounds of the current invention are capable of modulating glucocorticoid receptor activity by selectively binding to the GR or by preventing GR ligands from binding to the GR. In some embodiments, the compounds exhibit little or no cytotoxic effect.

A. Binding Assays

In some embodiments, GR modulators are identified by screening for molecules that compete with a ligand of GR, such as dexamethasone. Those of skill in the art will recognize that there are a number of ways to perform competitive binding assays. In some embodiments, GR is pre-incubated with a labeled GR ligand and then contacted with a test compound. This type of competitive binding assay may also be referred to herein as a binding displacement assay. Alteration (e.g., a decrease) of the quantity of ligand bound to GR indicates that the molecule is a potential GR modulator. Alternatively, the binding of a test compound to GR can be measured directly with a labeled test compound. This latter type of assay is called a direct binding assay.

Both direct binding assays and competitive binding assays can be used in a variety of different formats. The formats may be similar to those used in immunoassays and receptor binding assays. For a description of different formats for binding assays, including competitive binding assays and direct binding assays, see Basic and Clinical Immunology 7th Edition (D. Stites and A. Terr ed.) 1991; Enzyme Immunoassay, E. T. Maggio, ed., CRC Press, Boca Raton, Fla. (1980); and “Practice and Theory of Enzyme Immunoassays,” P. Tijssen, Laboratory Techniques in Biochemistry and Molecular Biology, Elsevier Science Publishers B.V. Amsterdam (1985), each of which is incorporated herein by reference.

In solid phase competitive binding assays, for example, the sample compound can compete with a labeled analyte for specific binding sites on a binding agent bound to a solid surface. In this type of format, the labeled analyte can be a GR ligand and the binding agent can be GR bound to a solid phase. Alternatively, the labeled analyte can be labeled GR and the binding agent can be a solid phase GR ligand. The concentration of labeled analyte bound to the capture agent is inversely proportional to the ability of a test compound to compete in the binding assay.

Alternatively, the competitive binding assay may be conducted in liquid phase, and any of a variety of techniques known in the art may be used to separate the bound labeled protein from the unbound labeled protein. For example, several procedures have been developed for distinguishing between bound ligand and excess bound ligand or between bound test compound and the excess unbound test compound. These include identification of the bound complex by sedimentation in sucrose gradients, gel electrophoresis, or gel isoelectric focusing; precipitation of the receptor-ligand complex with protamine sulfate or adsorption on hydroxylapatite; and the removal of unbound compounds or ligands by adsorption on dextran-coated charcoal (DCC) or binding to immobilized antibody. Following separation, the amount of bound ligand or test compound is determined.

Alternatively, a homogenous binding assay may be performed in which a separation step is not needed. For example, a label on the GR may be altered by the binding of the GR to its ligand or test compound. This alteration in the labeled GR results in a decrease or increase in the signal emitted by label, so that measurement of the label at the end of the binding assay allows for detection or quantitation of the GR in the bound state. A wide variety of labels may be used. The component may be labeled by any one of several methods. Useful radioactive labels include those incorporating ³H, ¹²⁵I, ³⁵S, ¹⁴C, or ³²P. Useful non-radioactive labels include those incorporating fluorophores, chemiluminescent agents, phosphorescent agents, electrochemiluminescent agents, and the like. Fluorescent agents are especially useful in analytical techniques that are used to detect shifts in protein structure such as fluorescence anisotropy and/or fluorescence polarization. The choice of label depends on sensitivity required, ease of conjugation with the compound, stability requirements, and available instrumentation. For a review of various labeling or signal producing systems which may be used, see U.S. Pat. No. 4,391,904, which is incorporated herein by reference in its entirety for all purposes. The label may be coupled directly or indirectly to the desired component of the assay according to methods well known in the art.

High-throughput screening methods may be used to assay a large number of potential modulator compounds. Such “compound libraries” are then screened in one or more assays, as described herein, to identify those library members (particular chemical species or subclasses) that display a desired characteristic activity. Preparation and screening of chemical libraries is well known to those of skill in the art. Devices for the preparation of chemical libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

B. Cell-Based Assays

Cell-based assays involve whole cells or cell fractions containing GR to assay for binding or modulation of activity of GR by a compound of the present invention. Exemplary cell types that can be used according to the methods of the invention include, e.g., any mammalian cells including leukocytes such as neutrophils, monocytes, macrophages, eosinophils, basophils, mast cells, and lymphocytes, such as T cells and B cells, leukemias, Burkitt's lymphomas, tumor cells (including mouse mammary tumor virus cells), endothelial cells, epithelial cells, fibroblasts, cardiac cells, muscle cells, breast tumor cells, ovarian cancer carcinomas, cervical carcinomas, glioblastomas, liver cells, kidney cells, and neuronal cells, as well as fungal cells, including yeast. Cells can be primary cells or tumor cells or other types of immortal cell lines. Of course, GR can be expressed in cells that do not express an endogenous version of GR.

In some cases, fragments of GR, as well as protein fusions, can be used for screening. When molecules that compete for binding with GR ligands are desired, the GR fragments used are fragments capable of binding the ligands (e.g., dexamethasone). Alternatively, any fragment of GR can be used as a target to identify molecules that bind GR. GR fragments can include any fragment of, e.g., at least 20, 30, 40, 50 amino acids up to a protein containing all but one amino acid of GR.

In some embodiments, signaling triggered by GR activation is used to identify GR modulators. Signaling activity of GR can be determined in many ways. For example, downstream molecular events can be monitored to determine signaling activity. Downstream events include those activities or manifestations that occur as a result of stimulation of a GR receptor. Exemplary downstream events useful in the functional evaluation of transcriptional activation and antagonism in unaltered cells include upregulation of a number of glucocorticoid response element (GRE)-dependent genes (PEPCK, tyrosine amino transferase, aromatase). In addition, specific cell types susceptible to GR activation may be used, such as osteocalcin expression in osteoblasts which is downregulated by glucocorticoids; primary hepatocytes which exhibit glucocorticoid mediated upregulation of PEPCK and glucose-6-phosphate (G-6-Pase)). GRE-mediated gene expression has also been demonstrated in transfected cell lines using well-known GRE-regulated sequences (e.g. the mouse mammary tumor virus promoter (MMTV) transfected upstream of a reporter gene construct). Examples of useful reporter gene constructs include luciferase (luc), alkaline phosphatase (ALP) and chloramphenicol acetyl transferase (CAT). The functional evaluation of transcriptional repression can be carried out in cell lines such as monocytes or human skin fibroblasts. Useful functional assays include those that measure IL-1beta or TNFα stimulated IL-6 expression; the downregulation of collagenase, cyclooxygenase-2 and various chemokines (MCP-1, RANTES); LPS stimulated cytokine release, e.g., TNFα; or expression of genes regulated by NFkB or AP-1 transcription factors in transfected cell-lines.

Typically, compounds that are tested in whole-cell assays are also tested in a cytotoxicity assay. Cytotoxicity assays are used to determine the extent to which a perceived modulating effect is due to non-GR binding cellular effects. In an exemplary embodiment, the cytotoxicity assay includes contacting a constitutively active cell with the test compound. Any decrease in cellular activity indicates a cytotoxic effect.

C. Specificity

The compounds of the present invention may be subject to a specificity assay (also referred to herein as a selectivity assay). Typically, specificity assays include testing a compound that binds GR in vitro or in a cell-based assay for the degree of binding to non-GR proteins. Selectivity assays may be performed in vitro or in cell based systems, as described above. Binding may be tested against any appropriate non-GR protein, including antibodies, receptors, enzymes, and the like. In an exemplary embodiment, the non-GR binding protein is a cell-surface receptor or nuclear receptor. In another exemplary embodiment, the non-GR protein is a steroid receptor, such as estrogen receptor, progesterone receptor, androgen receptor, or mineralocorticoid receptor.

The terms and expressions which have been employed herein are used as terms of description and not of limitation, and there is no intention in the use of such terms and expressions of excluding equivalents of the features shown and described, or portions thereof, it being recognized that various modifications are possible within the scope of the invention claimed. Moreover, any one or more features of any embodiment of the invention may be combined with any one or more other features of any other embodiment of the invention, without departing from the scope of the invention. For example, the features of the GR modulator compounds are equally applicable to the methods of treating disease states and/or the pharmaceutical compositions described herein. All publications, patents, and patent applications cited herein are hereby incorporated by reference in their entirety for all purposes.

### VII. Examples

¹H NMR spectra were recorded at ambient temperature using a Varian Unity Inova spectrometer (400 MHz) with a 5 mm inverse detection triple resonance probe for detection of H1, C13 and P31 or a Bruker Avance DRX spectrometer (400 MHz) with a 5 mm inverse detection triple resonance TXI probe, or a Bruker Avance III spectrometer (400 MHz).

LCMS was determined by one of the following methods, or by another method.

Method A: experiments were performed using a Waters Platform LC quadrupole mass spectrometer with positive and negative ion electrospray and ELS/Diode array detection using a Phenomenex Luna 3 micron C18 (2) 30×4.6 mm column and a 2 mL/minute flow rate. The solvent system was a 95% water containing 0.1% formic acid (solvent A) and a 5% acetonitrile containing 0.1% formic acid (solvent B) for the first 50 seconds followed by a gradient up to 5% solvent A and 95% solvent B over the next 4 minutes. The final solvent system was held constant for a further 1 minute.

Method B: experiments were performed using a Waters Micromass ZQ2000 quadrupole mass spectrometer with a positive and negative ion electrospray and ELS/Diode array detection using a Higgins Clipeus 5 micron C18 100×3.0 mm column and a 1 mL/minute flow rate. The initial solvent system was 95% water containing 0.1% formic acid (solvent A) and a 5% acetonitrile containing 0.1% formic acid (solvent B) for the first minute followed by a gradient up to 5% solvent A and 95% solvent B over the next 8 minutes. The final solvent system was held constant for a further 5 minutes.

Method C: experiments were performed using a Waters ZMD quadrupole mass spectrometer with positive and negative ion electrospray and ELS/Diode array detection using a Phenomenex Luna 3 micron C18 (2) 30×4.6 mm column and a 2 mL/minute flow rate. The solvent system was a 95% water containing 0.1% formic acid (solvent A) and a 5% acetonitrile containing 0.1% formic acid (solvent B) for the first 50 seconds followed by a gradient up to 5% solvent A and 95% solvent B over the next 4 minutes. The final solvent system was held constant for a further 1 minute.

Method D: experiments were performed using a Waters Micromass ZQ2000 quadrupole mass spectrometer linked to a Waters Acquity UPLC system with a PDA UV detector using an Acquity UPLC BEH C18 1.7 micron 100×2.1 mm, maintained at 40° C. The spectrometer has an electrospray source operating in positive and negative ion mode. The initial solvent system was 95% water containing 0.1% formic acid (solvent A) and a 5% acetonitrile containing 0.1% formic acid (solvent B) for 0.4 minutes followed by a gradient up to 5% solvent A and 95% solvent B over the next 6.4 minutes.

Method E: experiments were performed using a Waters Quattro Micro triple quadrupole mass spectrometer linked to a Hewlett Packard HP1100 LC system with a positive and negative ion electrospray and ELS/Diode array detection using a Higgins Clipeus 5 micron C18 100×3.0 mm column and a 1 mL/minute flow rate. The initial solvent system was 85% water containing 0.1% formic acid (solvent A) and 15% acetonitrile containing 0.1% formic acid (solvent B) for the first minute followed by a gradient up to 5% solvent A and 95% solvent B over the next 13 minutes. The solvent system was held constant for a further 7 minutes before returning to the initial solvent conditions.

Method F: experiments were performed using an Agilent Infinity 1260 LC 6120 quadrupole mass spectrometer with positive and negative ion electrospray and ELS/UV @ 254 nm detection using an Agilent Zorbax Extend C18, Rapid Resolution HT 1.8 micron C18 30×4.6 mm column and a 2.5 mL/minute flow rate. The initial solvent system was 95% water containing 0.1% formic acid (solvent A) and 5% acetonitrile containing 0.1% formic acid (solvent B) ramping up to 5% solvent A and 95% solvent B over the next 3.0 minutes, the flow rate was then increased to 4.5 mL/minute and held for 0.5 minutes at 95% B. Over 0.1 minute the gradient returned to 95% A and 5% B and 3.5 mL/minute and was held at these conditions for 0.3 minutes, the final 0.1 minute saw the return to the initial starting conditions, 95% A 5% B at 2.5 mL/minute.

### Example 1

### (R)-4a-Cyclopropylmethoxymethyl-1-(4-fluorophenyl)-6-[3-((R)-3-fluoropyrrolidin-1-yl)-benzenesulfonyl]-4,4a,5,6,7,8-hexahydro-1H-1,2,6-triaza-cyclopenta[b]naphthalene

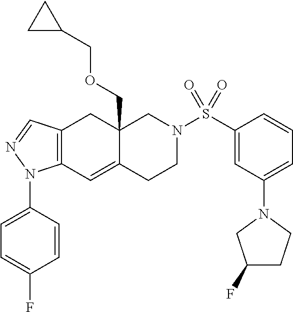 [SMILES: O=S(=O)(c1cccc(N2CC[C@@H](F)C2)c1)N1CCC2=Cc3c(cnn3-c3ccc(F)cc3)C[C@]2(COCC2CC2)C1]

A mixture of BINAP (2,2′-bis(diphenylphosphino)-1,1′-binaphthyl) (27.5 mg), tris(dibenzylideneacetone)dipalladium (20 mg), sodium tert-butoxide (91 mg), (R)-3-fluoropyrrolidine hydrochloride (69 mg) and (R)-6-(3-bromobenzenesulfonyl)-4a-cyclopropylmethoxymethyl-1-(4-fluorophenyl)-4,4a,5,6,7,8-hexahydro-1H-1,2,6-triaza-cyclopenta[b]naphthalene (125 mg) in dry tetrahydrofuran (4.5 ml) was heated in a microwave at 100° C. for 30 minutes. The resultant mixture was filtered through celite and the solvent was evaporated to provide an orange foam. This material was purified using a 12 g RediSep cartridge eluting with ethyl acetate in cyclohexane (0-40%) to give a yellow foam. Further purification was achieved by preparative HPLC eluting with a mixture of water and acetonitrile (60-90% acetonitrile) to provide the title compound as a fluffy white powder (80 mg). LCMS: 581 (M+H), retention time 5.89 minutes. ¹H NMR (400 MHz, CHCl₃-d): δ 7.39-7.31 (m, 4H), 7.14-7.09 (m, 2H), 7.05 (d, 1H, J=8.4 Hz), 6.88 (t, 1H, J=2.0 Hz), 6.70 (dd, 1H, J=8.3, 2.5 Hz), 6.22 (d, 1H, J=2.2 Hz), 5.38 (dt, 1H, J=53.5, 3.4 Hz), 4.16 (dd, 1H, J=11.5, 1.9 Hz), 3.89-3.82 (m, 1H), 3.66-3.56 (m, 1H), 3.55-3.43 (m, 4H), 3.33-3.26 (m, 2H), 3.18-3.11 (m, 2H), 2.74-2.63 (m, 1H), 2.45-2.29 (m, 3H), 2.26-2.06 (m, 3H), 1.07-0.97 (m, 1H), 0.51-0.42 (m, 2H), 0.28-0.11 (m, 2H).

### Preparation 1a. (R)-6-(3-Bromobenzenesulfonyl)-4a-cyclopropylmethoxymethyl-1-(4-fluorophenyl)-4,4a,5,6,7,8-hexahydro-1H-1,2,6-triaza-cyclopenta[b]naphthalene

To a solution of (R)-6-(3-bromobenzenesulfonyl)-1-(4-fluorophenyl)-4a-hydroxymethyl-4,4a,5,6,7,8-hexahydro-1H-1,2,6-triaza-cyclopenta[b]naphthalene (1.3 g) in tetrahydrofuran (20 ml) was added 50% aqueous sodium hydroxide solution (12.5 molar, 10 ml) followed by tetrabutylammoniumhydrogensulfate (424 mg), tetrabutylammonium iodide (1.85 g) and cyclopropylmethyl bromide (4.85 ml). The reaction mixture was heated at 40° C. for 7 hours. A further quantity of tetrabutyl ammonium hydrogensulfate (424 mg), tetrabutyl ammonium iodide (1.85 g) and cyclopropylmethyl bromide (4.85 ml) was added and the reaction mixture was stirred at 40° C. for 16 hours. After dilution with water (50 ml) the mixture was extracted with ethyl acetate and the combined organic extracts were washed with saturated aqueous sodium chloride solution, dried over sodium sulfate, filtered and concentrated under vacuum. The crude product was purified on a 40 g RediSep cartridge eluting with ethyl acetate in cyclohexane (0-30%). The title compound was obtained as an off-white foam (1.21 g), which was used without any further purification.

### Preparation 1b. (R)-6-(3-Bromobenzenesulfonyl)-1-(4-fluorophenyl)-4a-hydroxymethyl-4,4a,5,6,7,8-hexahydro-1H-1,2,6-triaza-cyclopenta[b]naphthalene

(R)-6-(3-Bromobenzenesulfonyl)-1-(4-fluoro-phenyl)-benzenesulfonyl]-1,4,5,6,7,8-hexahydro-1,2-6-triaza-cyclopenta[b]naphthalene-4a-carboxylic acid methyl ester (2.5 g) was dissolved in dry dichloromethane (60 ml) and the solution cooled to −78° C. under nitrogen. Diisobutylaluminiumhydride (1 molar solution in toluene, 18.3 ml) was added dropwise whilst maintaining the reaction temperature below −70° C. The reaction mixture was stirred at −78° C. for 2 hours and then allowed to warm to 10° C. and quenched by the careful addition of water (6.5 ml). Ethyl acetate was added, followed by sodium hydrogen carbonate, and the resultant mixture was stirred for 10 minutes to provide a granular precipitate. Sodium sulfate was added, and after stirring, the mixture was filtered through celite to remove solids. The filtrate was concentrated under vacuum to provide the title compound as an off-white solid (2.25 g). LCMS: 519 (M+1), retention time 3.82 minutes.

### Preparation 1c. (R)-6-(3-Bromobenzenesulfonyl)-1-(4-fluoro-phenyl)-benzenesulfonyl]-1,4,5,6,7,8-hexahydro-1,2-6-triaza-cyclopenta[b]naphthalene-4a-carboxylic acid methyl ester

A solution of (R)-1-(4-fluorophenyl)-1,4,7,8-tetrahydro-1,2,6-triazacyclopenta[b]naphthalene-4a,6-dicarboxylic acid 6-tert-butyl ester 4a-methyl ester (2.1 g) in 4 molar hydrochloric acid in dioxane (50 ml) was stirred for 75 minutes and then the mixture was concentrated under vacuum. The resultant gum was co-evaporated with diethyl ether to provide a sticky orange gum. This crude material was dissolved in dichloromethane (30 ml) and diisopropylethylamine (4.29 ml) and treated with 3-bromobenzenesulfonyl chloride (1.51 g). After stirring at room temperature for 16 hours, the reaction mixture was diluted with dichloromethane and then washed with saturated aqueous sodium hydrogen carbonate solution, followed by saturated aqueous sodium chloride solution. The combined organic extracts were dried over sodium sulfate, filtered and concentrated under vacuum to give an orange/brown sticky gum. This crude material was purified on an 80 g RediSep cartridge eluting with ethyl acetate in cyclohexane (0-40%). The title compound was obtained as a cream coloured foam (2.55 g). LCMS: 546 (M+1), retention time 4.04 minutes.

### Example 2

### (R)-4a-Cyclopropylmethoxymethyl-1-(4-fluorophenyl)-6-[3-((S)-3-fluoropyrrolidin-1-yl)-phenyl-3-sulfonyl]-4,4a,5,6,7,8-hexahydro-1H-1,2,6-triaza-cyclopenta[b]naphthalene

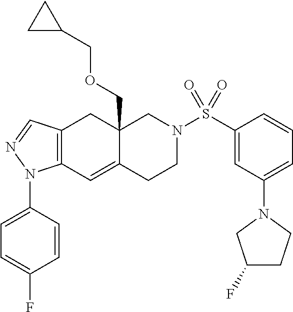 [SMILES: O=S(=O)(c1cccc(N2CC[C@H](F)C2)c1)N1CCC2=Cc3c(cnn3-c3ccc(F)cc3)C[C@]2(COCC2CC2)C1]

The title compound was prepared by the method of Example 1 using (S)-3-fluoropyrrolidine hydrochloride. LCMS 581 (M+1), retention time 5.89 minutes. ¹H NMR (400 MHz, CHCl₃-d): δ 7.40-7.31 (m, 4H), 7.15-7.08 (m, 2H), 7.05 (d, 1H, J=7.6 Hz), 6.88 (t, 1H, J=2.1 Hz), 6.70 (dd, 1H, J=8.3, 2.5 Hz), 6.22 (d, 1H, J=2.3 Hz), 5.38 (dt, 1H, J=53.6, 3.3 Hz), 4.16 (dd, 1H, J=11.5, 2.0 Hz), 3.90-3.82 (m, 1H), 3.66-3.55 (m, 1H), 3.55-3.42 (m, 4H), 3.34-3.25 (m, 2H), 3.18-3.10 (m, 2H), 2.74-2.63 (m, 1H), 2.45-2.29 (m, 3H), 2.26-2.05 (m, 3H), 1.07-0.97 (m, 1H), 0.53-0.42 (m, 2H), 0.26-0.13 (m, 2H).

### Example 3

### (R)-4a-Cyclopropylmethoxymethyl-1-(4-fluorophenyl)-6-[3-(pyrrolidin-1-yl)-benzenesulfonyl]-4,4a,5,6,7,8-hexahydro-1H-1,2,6-triaza-cyclopenta[b]naphthalene

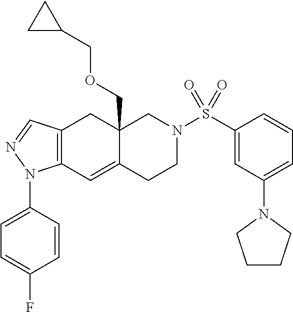 [SMILES: O=S(=O)(c1cccc(N2CCCC2)c1)N1CCC2=Cc3c(cnn3-c3ccc(F)cc3)C[C@]2(COCC2CC2)C1]

The title compound was prepared by the method of Example 1 using pyrrolidine. LCMS: 563 (M+1), retention time 6.33 minutes. ¹H NMR (400 MHz, CHCl₃-d): δ 7.43-7.38 (m, 3H), 7.35-7.31 (m, 1H), 7.18-7.11 (m, 2H), 7.02 (d, 1H, J=7.6 Hz), 6.88 (t, 1H, J=2.2 Hz), 6.71 (dd, 1H, J=8.4, 2.6 Hz), 6.25 (d, 1H, J=2.4 Hz), 4.19 (dd, 1H, J=11.4, 2.0 Hz), 3.92-3.86 (m, 1H), 3.48 (d, 1H, J=9.0 Hz), 3.36-3.28 (m, 6H), 3.20 (d, 1H, J=7.3 Hz), 3.16 (d, 1H, J=15.8 Hz), 2.76-2.66 (m, 1H), 2.42-2.32 (m, 2H), 2.19 (d, 1H, J=15.8 Hz), 2.13 (d, 1H, J=11.3 Hz), 2.07-2.00 (m, 4H), 1.10-1.00 (m, 1H), 0.52-0.47 (m, 2H), 0.29-0.14 (m, 2H).

### Example 4

### (R)-1-(4-Fluorophenyl)-6-[3-((R)-3-fluoropyrrolidin-1-yl)-benzenesulfonyl]-4a-methoxymethyl-4,4a,5,6,7,8-hexahydro-1H-1,2,6-triaza-cyclopenta[b]naphthalene

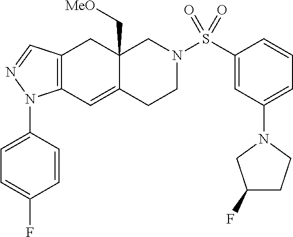 [SMILES: COC[C@]12Cc3cnn(-c4ccc(F)cc4)c3C=C1CCN(S(=O)(=O)c1cccc(N3CC[C@@H](F)C3)c1)C2]

The title compound was prepared by the method of Example 1 using (R)-3-fluoropyrrolidine and (R)-6-(3-bromobenzenesulfonyl)-1-(4-fluorophenyl)-4a-methoxymethyl-4,4a,5,6,7,8-hexahydro-1H-1,2,6-triaza-cyclopenta[b]naphthalene. LCMS: 541 (M+H), retention time 5.57 minutes. ¹H NMR (400 MHz, CHCl₃-d): δ 7.40-7.30 (m, 4H), 7.15-7.08 (m, 2H), 7.05 (d, 1H, J=7.8 Hz), 6.88 (t, 1H, J=2.0 Hz), 6.70 (dd, 1H, J=8.3, 2.3 Hz), 6.24 (d, 1H, J=2.2 Hz), 5.38 (1H, dt, J=53.5, 3.4 Hz), 4.12 (dd, 1H, J=11.5, 1.8 Hz), 3.91-3.84 (m, 1H), 3.66-3.55 (m, 1H), 3.55-3.44 (m, 3H), 3.39 (d, 1H, J=9.1 Hz), 3.34 (s, 3H), 3.10 (d, 1H, J=9.1 Hz), 3.06 (d, 1H, J=15.7 Hz), 2.73-2.63 (m, 1H), 2.45-2.29 (m, 3H), 2.26-2.05 (m, 3H).

### Preparation 4a. (R)-6-(3-Bromobenzenesulfonyl)-1-(4-fluorophenyl)-4a-methoxymethyl-4,4a,5,6,7,8-hexahydro-1H-1,2,6-triaza-cyclopenta[b]naphthalene

(R)-6-(3-Bromobenzenesulfonyl)-1-(4-fluorophenyl)-4a-hydroxymethyl-4,4a,5,6,7,8-hexahydro-1H-1,2,6-triaza-cyclopenta[b]naphthalene (1 g) was dissolved in dry tetrahydrofuran (30 ml) under argon. Sodium hydride (60% dispersion in oil, 232 mg) was added portion-wise and the reaction mixture was stirred for 45 minutes at room temperature. Iodomethane (361 μl) was added and the resultant mixture was stirred at 45° C. for 18 hours. The reaction mixture was cooled to room temperature and diluted with water (50 ml), then extracted with ethyl acetate. The combined organic extracts were washed with saturated aqueous sodium chloride solution, then dried over sodium sulphate, filtered and concentrated under vacuum to provide a yellow foam. The crude material was purified on a 24 g RediSep cartridge eluting with ethyl acetate in cyclohexane (0-35%). The title compound was obtained as a white foam (860 mg), and used without further purification.

### Example 5

### (R)-1-(4-Fluorophenyl)-6-[3-((S)-3-fluoropyrrolidin-1-yl)-benzenesulfonyl]-4a-methoxymethyl-4,4a,5,6,7,8-hexahydro-1H-1,2,6-triaza-cyclopenta[b]naphthalene

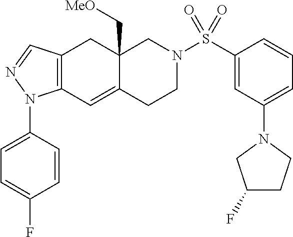 [SMILES: COC[C@]12Cc3cnn(-c4ccc(F)cc4)c3C=C1CCN(S(=O)(=O)c1cccc(N3CC[C@H](F)C3)c1)C2]

The title compound was prepared by the method of Example 1 using (S)-3-fluoropyrrolidine hydrochloride and (R)-6-(3-bromobenzenesulfonyl)-1-(4-fluorophenyl)-4a-methoxymethyl-4,4a,5,6,7,8-hexahydro-1H-1,2,6-triaza-cyclopenta[b]naphthalene. LCMS: 541 (M+1), retention time 5.51 minutes. ¹H NMR (400 MHz, CHCl₃-d): δ 7.40-7.30 (m, 4H), 7.15-7.08 (m, 2H), 7.05 (d, 1H, J=8.0 Hz), 6.88 (t, 1H, J=2.2 Hz), 6.70 (dd, 1H, J=8.1, 2.3 Hz), 6.23 (d, 1H, J=2.3 Hz), 5.38 (dt, 1H, J=53.7, 3.7 Hz), 4.12 (dd, 1H, J=11.6, 2.0 Hz), 3.91-3.85 (m, 1H), 3.66-3.55 (m, 1H), 3.54-3.51 (m, 1H), 3.51-3.44 (m, 2H), 3.40 (d, 1H, J=9.1 Hz), 3.34 (s, 3H), 3.10 (d, 1H, J=8.8 Hz), 3.06 (d, 1H, J=15.9 Hz), 2.74-2.63 (m, 1H), 2.45-2.30 (m, 3H), 2.26-2.05 (m, 3H).

### Example 6

### (R)-1-(4-Fluoro-phenyl)-4a-methoxymethyl-6-[3-(pyrrolidin-1-yl)-benzenesulfonyl)-4,4a,5,6,7,8-hexahydro-1H-1,2,6-triaza-cyclopenta[b]naphthalene

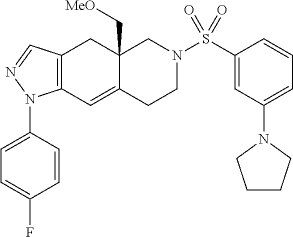 [SMILES: COC[C@]12Cc3cnn(-c4ccc(F)cc4)c3C=C1CCN(S(=O)(=O)c1cccc(N3CCCC3)c1)C2]

The title compound was prepared by the method of Example 1 using pyrrolidine and (R)-6-(3-bromo-benzenesulfonyl)-1-(4-fluoro-phenyl)-4a-methoxymethyl-4,4a,5,6,7,8-hexahydro-1H-1,2,6-triaza-cyclopenta[b]naphthalene. LCMS: 523 (M+1), retention time 5.92 minutes. ¹H NMR (400 MHz, CHCl₃-d): δ 7.39-7.34 (m, 3H), 7.29 (t, 1H, J=8.1 Hz), 7.14-7.08 (m, 2H), 6.97 (d, 1H, J=7.8 Hz), 6.85 (t, 1H, J=2.3 Hz), 6.67 (dd, 1H, J=8.4, 2.6 Hz), 6.22 (d, 1H, J=2.3 Hz), 4.12 (dd, 1H, J=11.5, 2.0 Hz), 3.91-3.84 (m, 1H), 3.40 (d, 1H, J=9.0 Hz), 3.35 (s, 3H), 3.31-3.26 (m, 4H), 3.10 (d, 1H, J=9.1 Hz), 3.06 (d, 1H, J=15.8 Hz), 2.73-2.63 (m, 1H), 2.41-2.29 (m, 2H), 2.16 (d, 1H, J=15.8 Hz), 2.10 (d, 1H, J=11.6 Hz), 2.04-1.99 (m, 4H).

### Example 7

### (R)-4a-Cyclopropylmethoxymethyl-1-(4-fluoro-phenyl)-6-[4-((R)-3-fluoro-pyrrolidin-1-yl)-benzenesulfonyl]-4,4a,5,6,7,8-hexahydro-1H-1,2,6-triaza-cyclopenta[b]naphthalene

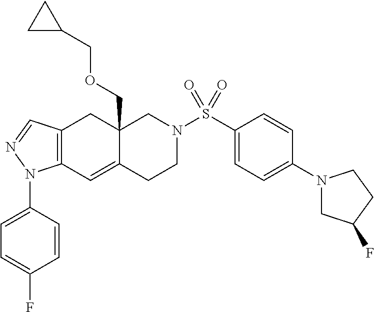 [SMILES: O=S(=O)(c1ccc(N2CC[C@@H](F)C2)cc1)N1CCC2=Cc3c(cnn3-c3ccc(F)cc3)C[C@]2(COCC2CC2)C1]

The title compound was prepared by the method of Example 1 using (R)-3-fluoropyrrolidine hydrochloride and (R)-6-(4-bromo-benzenesulfonyl)-4a-cyclopropylmethoxymethyl-1-(4-fluoro-phenyl)-4,4a,5,6,7,8-hexahydro-1H-1,2,6-triaza-cyclopenta[b]naphthalene. LCMS 581 (M+1), retention time 5.75 minutes. ¹H NMR (400 MHz, CHCl₃-d): δ 7.63-7.59 (m, 2H), 7.40-7.34 (m, 3H), 7.14-7.08 (m, 2H), 6.57-6.53 (m, 2H), 6.21 (d, 1H, J=2.2 Hz), 5.37 (d, 1H, J=52.8 Hz), 4.12 (dd, 1H, J=11.3, 2.0 Hz), 3.87-3.80 (m, 1H), 3.62 (d, 1H, J=1.9 Hz), 3.56-3.48 (m, 3H), 3.42 (d, 1H, J=9.1 Hz), 3.29 (d, 2H, J=6.7 Hz), 3.16 (d, 1H, J=9.9 Hz), 3.12 (d, 1H, J=15.9 Hz), 2.72-2.62 (m, 1H), 2.46-2.04 (m, 5H), 2.00 (d, 1H, J=11.3 Hz), 1.06-0.95 (m, 1H), 0.50-0.41 (m, 2H), 0.26-0.13 (m, 2H).

### Preparation 7a. (R)-6-(4-Bromo-benzenesulfonyl)-4a-cyclopropylmethoxymethyl-1-(4-fluoro-phenyl)-4,4a,5,6,7,8-hexahydro-1H-1,2,6-triaza-cyclopenta[b]naphthalene

Trifluoroacetic acid (5.3 ml) was added to (R)-4a-cyclopropylmethoxymethyl-1-(4-fluoro-phenyl)-1,4,4a,5,7,8-hexahydro-1,2,6-triaza-cyclopenta[b]naphthalene-6-carboxylic acid tert-butyl ester (673 mg) and the resultant mixture was stirred at room temperature for 30 minutes. The mixture was concentrated under vacuum and co-evaporated with diethyl ether to provide a yellow solid. This solid was dissolved in dichloromethane (7.5 ml) and 4-bromo-benzenesulfonyl chloride (436 mg) and diisopropylethylamine (1.3 mL) were added. The reaction mixture was stirred for 2 hours and then diluted with dichloromethane (400 mL). After washing with saturated aqueous sodium hydrogen carbonate solution, the mixture was filtered and then concentrated under vacuum. Purification by flash chromatography eluting with ethyl acetate in cyclohexane (0-45%) provided the title compound (829 mg). LCMS: 573 (M+1), retention time 4.48 minutes.

### Preparation 7b. (R)-4a-Cyclopropylmethoxymethyl-1-(4-fluoro-phenyl)-1,4,4a,5,7,8-hexahydro-1,2,6-triaza-cyclopenta[b]naphthalene-6-carboxylic acid tert-butyl ester

50% aqueous sodium hydroxide solution (11 ml) and cyclopropylmethyl bromide (805 μl) were added sequentially to a mixture of (R)-1-(4-fluoro-phenyl)-4a-hydroxymethyl-1,4,4a,5,7,8-hexahydro-1,2,6-triaza-cyclopenta[b]naphthalene-6-carboxylic acid tert-butyl ester (1.1 g), tetrabutylammonium hydrogen sulfate (934 mg) and tetrabutylammonium iodide (4.06 g) in tetrahydrofuran (25 ml). The resultant mixture was warmed to 45° C. under argon and stirred for 16 hours. After cooling to room temperature the mixture was treated with water and then extracted with ethyl acetate. The combined organic extracts were washed with water and saturated aqueous sodium chloride solution, dried over sodium sulfate, filtered and concentrated under vacuum. The residue was purified by flash chromatography eluting with ethyl acetate in cyclohexane (0-70%) to provide the title compound (682 mg). LCMS: 454 (M+1), retention time 4.46 minutes.

### Example 8

### (R)-4a-Cyclopropylmethoxymethyl-1-(4-fluoro-phenyl)-6-[4-(pyrrolidin-1-yl)-benzenesulfonyl]-4,4a,5,6,7,8-hexahydro-1H-1,2,6-triaza-cyclopenta[b]naphthalene

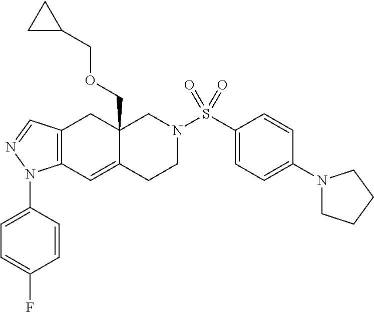 [SMILES: O=S(=O)(c1ccc(N2CCCC2)cc1)N1CCC2=Cc3c(cnn3-c3ccc(F)cc3)C[C@]2(COCC2CC2)C1]

The title compound was prepared by the method of Example 1 using pyrrolidine and (R)-6-(4-bromo-benzenesulfonyl)-4a-cyclopropylmethoxymethyl-1-(4-fluoro-phenyl)-4,4a,5,6,7,8-hexahydro-1H-1,2,6-triaza-cyclopenta[b]naphthalene. LCMS: 563 (M+1), retention time 6.15 minutes. ¹H NMR (400 MHz, CHCl₃-d): δ 7.60-7.56 (m, 2H), 7.40-7.34 (m, 3H), 7.14-7.08 (m, 2H), 6.54-6.50 (m, 2H), 6.21 (d, 1H, J=2.2 Hz), 4.12 (dd, 1H, J=11.4, 2.0 Hz), 3.87-3.80 (m, 1H), 3.44 (d, 1H, J=9.1 Hz), 3.34-3.27 (m, 6H), 3.16 (d, 1H, J=9.2 Hz), 3.12 (d, 1H, J=15.8 Hz), 2.72-2.61 (m, 1H), 2.34-2.22 (m, 2H), 2.14 (d, 1H, J=15.7 Hz), 2.05-1.97 (m, 5H), 1.06-0.95 (m, 1H), 0.50-0.41 (m, 2H), 0.26-0.13 (m, 2H).

### Example 9

### (R)-1-(4-Fluoro-phenyl)-6-[4-((R)-3-fluoro-pyrrolidin-1-yl)-benzenesulfonyl]-4a-methoxymethyl-4,4a,5,6,7,8-hexahydro-1H-1,2,6-triaza-cyclopenta[b]naphthalene

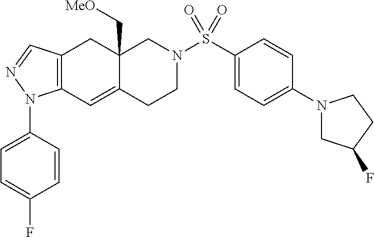 [SMILES: COC[C@]12Cc3cnn(-c4ccc(F)cc4)c3C=C1CCN(S(=O)(=O)c1ccc(N3CC[C@@H](F)C3)cc1)C2]

The title compound was prepared by the method of Example 1 using (R)-3-fluoropyrrolidine hydrochloride and (R)-6-(4-bromo-benzenesulfonyl)-1-(4-fluoro-phenyl)-4a-methoxymethyl-4,4a,5,6,7,8-hexahydro-1H-1,2,6-triaza-cyclopenta[b]naphthalene. LCMS: 541 (M+1), retention time 4.3 minutes. ¹H NMR (400 MHz, CHCl₃-d): δ 7.62-7.57 (m, 2H), 7.39-7.33 (m, 3H), 7.14-7.07 (m, 2H), 6.56-6.52 (m, 2H), 6.21 (d, 1H, J=2.3 Hz), 5.36 (d, 1H, J=53.2 Hz), 4.06 (dd, 1H, J=11.4, 1.9 Hz), 3.86-3.80 (m, 1H), 3.61 (d, 1H, J=1.9 Hz), 3.55-3.46 (m, 3H), 3.38 (d, 1H, J=9.1 Hz), 3.33 (s, 3H), 3.09 (d, 1H, J=9.1 Hz), 3.04 (d, 1H, J=15.8 Hz), 2.71-2.60 (m, 1H), 2.44-2.04 (m, 5H), 2.00 (d, 1H, J=11.3 Hz).

### Preparation 9a. (R)-6-(4-Bromo-benzenesulfonyl)-1-(4-fluoro-phenyl)-4a-methoxymethyl-4,4a,5,6,7,8-hexahydro-1H-1,2,6-triaza-cyclopenta[b]naphthalene

The title compound was prepared by the method of Preparation 1c from (R)-1-(4-fluoro-phenyl)-4a-methoxymethyl-1,4,4a,5,7,8-hexahydro-1,2,6-triaza-cyclopenta[b]naphthalene-6-carboxylic acid tert-butyl ester and 4-bromobenzenesulfonyl chloride. LCMS: 532 (M+1), retention time 4.3 minutes.

### Preparation 9b. (R)-1-(4-Fluoro-phenyl)-4a-methoxymethyl-1,4,4a,5,7,8-hexahydro-1,2,6-triaza-cyclopenta[b]naphthalene-6-carboxylic acid tert-butyl ester

The title compound was prepared by the method of Preparation 4a from (R)-1-(4-fluoro-phenyl)-4a-hydroxymethyl-1,4,4a,5,7,8-hexahydro-1,2,6-triaza-cyclopenta[b]naphthalene-6-carboxylic acid tert-butyl ester. LCMS: 414 (M+1), retention time 4.16 minutes.

### Example 10

### (R)-1-(4-Fluoro-phenyl)-6-[4-(pyrrolidin-1-yl)-benzenesulfonyl]-4a-methoxymethyl-4,4a,5,6,7,8-hexahydro-1H-1,2,6-triaza-cyclopenta[b]naphthalene

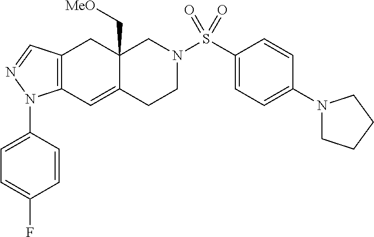 [SMILES: COC[C@]12Cc3cnn(-c4ccc(F)cc4)c3C=C1CCN(S(=O)(=O)c1ccc(N3CCCC3)cc1)C2]

The title compound was prepared by the method of Example 1 using pyrrolidine and (R)-6-(4-bromo-benzenesulfonyl)-1-(4-fluoro-phenyl)-4a-methoxymethyl-4,4a,5,6,7,8-hexahydro-1H-1,2,6-triaza-cyclopenta[b]naphthalene. LCMS: 523 (M+1), retention time 4.25 minutes. ¹H NMR (400 MHz, CHCl₃-d): δ 7.59-7.55 (m, 2H), 7.39-7.34 (m, 3H), 7.14-7.07 (m, 2H), 7.54-7.49 (m, 2H), 6.21 (d, 1H, J=2.2 Hz), 4.06 (dd, 1H, J=11.3, 2.0 Hz), 3.87-3.81 (m, 1H), 3.39 (d, 1H, J=9.0 Hz), 3.34 (s, 3H), 3.33-3.28 (m, 4H), 3.10 (d, 1H, J=9.0 Hz), 3.05 (d, 1H, J=15.8 Hz), 2.72-2.61 (m, 1H), 2.34-2.23 (m, 2H), 2.14 (d, 1H, J=15.7 Hz), 2.04-1.97 (m, 5H).

### Example 11

### (R)-1-(4-Fluoro-phenyl)-6-[3-((R)-3-fluoro-pyrrolidin-1-yl)-benzenesulfonyl]-1,4,5,6,7,8-hexahydro-1,2,6-triaza-cyclopenta[b]naphthalene-4a-carboxylic acid ethyl ester

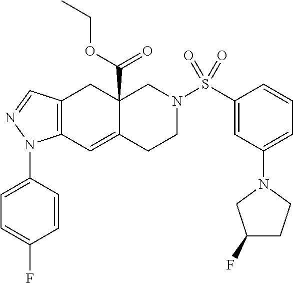 [SMILES: CCOC(=O)[C@]12Cc3cnn(-c4ccc(F)cc4)c3C=C1CCN(S(=O)(=O)c1cccc(N3CC[C@@H](F)C3)c1)C2]

Cesium carbonate (230 mg) followed by iodoethane (75 μl) were added to a solution of (R)-1-(4-fluoro-phenyl)-6-[3-((R)-3-fluoro-pyrrolidin-1-yl)-benzenesulfonyl]-1,4,5,6,7,8-hexahydro-1,2,6-triaza-cyclopenta[b]naphthalene-4a-carboxylic acid (127 mg) in N,N-dimethylformamide (2 ml). The resultant mixture was stirred at room temperature for 16 hours and then diluted with ethyl acetate (100 mL). After washing with water and saturated aqueous sodium chloride solution the solution was dried over sodium sulphate, filtered and concentrated under vacuum. Purification by flash chromatography eluting with ethyl acetate in cyclohexane (0-40%) and further purification by HPLC eluting with a mixture of acetonitrile and water containing 0.1% formic acid (40-85%) provided the title compound (48 mg). LCMS: 569 (M+1), retention time 5.42 minutes. ¹H NMR (400 MHz, CHCl₃-d): δ 7.41-7.30 (m, 4H), 7.16-7.09 (m, 2H), 7.01 (d, 1H, J=8.0 Hz), 6.86 (t, 1H, J=2.1 Hz), 6.70 (dd, 1H, J=8.2, 2.4 Hz), 6.36 (d, 1H, J=2.3 Hz), 5.37 (dt, 1H, J=53.5, 3.4 Hz), 4.36 (dd, 1H, J=11.7, 2.0 Hz), 4.14 (dq, 2H, J=7.2, 2.2 Hz), 3.88-3.81 (m, 1H), 3.66-3.56 (m, 1H), 3.54-3.52 (m, 1H), 3.51-3.45 (m, 2H), 3.27 (d, 1H, J=16.1 Hz), 2.94-2.84 (m, 1H), 2.54 (d, 1H, J=16.1 Hz), 2.46-2.34 (m, 4H), 2.26-2.05 (m, 1H), 1.23 (t, 3H, J=7.2 Hz).

### Preparation 11a. (R)-1-(4-Fluoro-phenyl)-6-[3-((R)-3-fluoro-pyrrolidin-1-yl)-benzenesulfonyl]-1,4,5,6,7,8-hexahydro-1,2,6-triaza-cyclopenta[b]naphthalene-4a-carboxylic acid

The title compound was prepared by the method of Example 1 using (R)-6-(3-bromo-benzenesulfonyl)-1-(4-fluoro-phenyl)-1,4,5,6,7,8-hexahydro-1,2,6-triazacyclopenta[b]naphthalene-4a-carboxylic acid methyl ester and (R)-3-fluoropyrrolidine hydrochloride. LCMS: 541 (M+1), retention time 3.6 minutes.

### Example 12

### (R)-1-(4-Fluoro-phenyl)-6-[3-((R)-3-fluoro-pyrrolidin-1-yl)-benzenesulfonyl]-1,4,5,6,7,8-hexahydro-1,2,6-triaza-cyclopenta[b]naphthalene-4a-carboxylic acid methyl ester

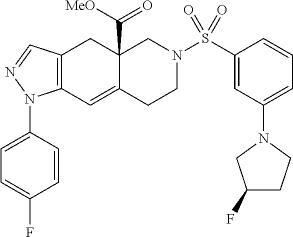 [SMILES: COC(=O)[C@]12Cc3cnn(-c4ccc(F)cc4)c3C=C1CCN(S(=O)(=O)c1cccc(N3CC[C@@H](F)C3)c1)C2]

The title compound was prepared by the method of Example 11 using iodomethane in place of iodoethane. LCMS: 555 (M+1), retention time 5.24 minutes. ¹H NMR (400 MHz, CHCl₃-d): δ 7.41-7.31 (m, 4H), 7.15-7.09 (m, 2H), 7.03 (d, 1H, J=8.0 Hz), 6.86 (t, 1H, J=2.2 Hz), 6.70 (dd, 1H, J=8.2, 2.5 Hz), 6.37 (d, 1H, J=2.3 Hz), 5.37 (dt, 1H, J=53.5, 3.4 Hz), 4.34 (dd, 1H, J=11.7, 1.9 Hz), 3.89-3.82 (m, 1H), 3.68 (s, 3H), 3.66-3.55 (m, 1H), 3.54-3.52 (m, 1H), 3.51-3.44 (m, 2H), 3.27 (d, 1H, J=16.2 Hz), 2.94-2.83 (m, 1H), 2.55 (d, 1H, J=16.2 Hz), 2.46-2.34 (m, 4H), 2.26-2.05 (m, 1H).

### Example 13

### (R)-1-(4-Fluoro-phenyl)-6-[3-((R)-3-fluoro-pyrrolidin-1-yl)-benzenesulfonyl]-1,4,5,6,7,8-hexahydro-1,2,6-triaza-cyclopenta[b]naphthalene-4a-carboxylic acid fluoromethyl ester

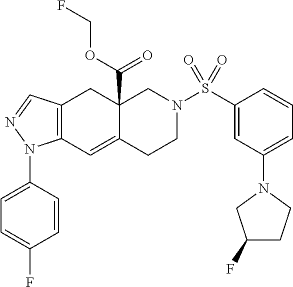 [SMILES: O=C(OCF)[C@]12Cc3cnn(-c4ccc(F)cc4)c3C=C1CCN(S(=O)(=O)c1cccc(N3CC[C@@H](F)C3)c1)C2]

A solution of (R)-1-(4-fluoro-phenyl)-6-[3-((R)-3-fluoro-pyrrolidin-1-yl)-benzenesulfonyl]-1,4,5,6,7,8-hexahydro-1,2,6-triaza-cyclopenta[b]naphthalene-4a-carboxylic acid (100 mg) in dry N,N-dimethylformamide (1.8 ml) was treated with bromofluoromethane (42 μl) followed by sodium carbonate (234 mg). The resultant mixture was stirred at room temperature for 16 hours and then diluted with ethyl acetate, washed with water and saturated aqueous sodium chloride solution. The organic extracts were dried over sodium sulfate, filtered and concentrated under vacuum. The crude product was purified by flash chromatography eluting with ethyl acetate in cyclohexane (0-40%) and then by reverse phase HPLC eluting with a mixture of acetonitrile and water containing 0.1% formic acid (50-90%) to provide the title compound (40 mg). LCMS: 573 (M+1), retention time 5.21 minutes. ¹H NMR (400 MHz, CHCl₃-d): δ 7.41-7.30 (m, 4H), 7.16-7.09 (m, 2H), 7.02 (d, 1H, J=7.8 Hz), 6.86 (t, 1H, J=2.2 Hz), 6.71 (dd, 1H, J=8.3, 2.5 Hz), 6.41 (d, 1H, J=2.2 Hz), 5.79 (dd, 1H, J=50.3, 1.8 Hz), 5.59 (dd, 1H, J=50.4, 1.8 Hz), 5.37 (dt, 1H, J=53.6, 3.6 Hz), 4.40 (dd, 1H, J=12.0, 2.2 Hz), 3.88-3.81 (m, 1H), 3.65-3.55 (m, 1H), 3.54-3.51 (m, 1H), 3.51-3.44 (m, 2H), 3.31 (d, 1H, J=16.4 Hz), 2.91-2.80 (m, 1H), 2.63 (d, 1H, J=16.4 Hz), 2.49-2.34 (m, 4H), 2.26-2.05 (m, 1H).

### Example 14

### (R)-1-(4-Fluoro-phenyl)-6-[3-(pyrrolidin-1-yl)-benzenesulfonyl]-1,4,5,6,7,8-hexahydro-1,2,6-triaza-cyclopenta[b]naphthalene-4a-carboxylic acid ethyl ester

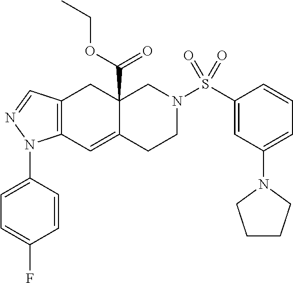 [SMILES: CCOC(=O)[C@]12Cc3cnn(-c4ccc(F)cc4)c3C=C1CCN(S(=O)(=O)c1cccc(N3CCCC3)c1)C2]

The title compound was prepared by the method of Example 11 from (R)-1-(4-fluoro-phenyl)-6-[3-(pyrrolidin-1-yl)-benzenesulfonyl]-1,4,5,6,7,8-hexahydro-1,2,6-triaza-cyclopenta[b]naphthalene-4a-carboxylic acid and iodoethane. LCMS: 551 (M+1), retention time 5.79 minutes. ¹H NMR (400 MHz, CHCl₃-d): δ 7.39-7.32 (m, 3H), 7.28 (t, 1H, J=7.8 Hz), 7.14-7.07 (m, 2H), 6.95 (d, 1H, J=7.8 Hz), 6.82 (t, 1H, J=2.1 Hz), 6.66 (dd, 1H, J=8.4, 2.3 Hz), 6.34 (d, 1H, J=2.2 Hz), 4.34 (dd, 1H, J=11.6, 2.0 Hz), 4.14 (dq, 2H, J=7.1, 2.8 Hz), 3.86-3.80 (m, 1H), 3.30-3.24 (m, 5H), 2.93-2.83 (m, 1H), 2.52 (d, 1H, J=16.1 Hz), 2.44-2.32 (m, 3H), 2.03=1.97 (m, 4H), 1.22 (t, 3H, J=7.1 Hz).

### Preparation 14a. (R)-1-(4-Fluoro-phenyl)-6-[3-(pyrrolidin-1-yl)-benzenesulfonyl]-1,4,5,6,7,8-hexahydro-1,2,6-triaza-cyclopenta[b]naphthalene-4a-carboxylic acid

The title compound was prepared by the method of Example 1 from (R)-6-(3-bromo-benzenesulfonyl)-1-(4-fluoro-phenyl)-1,4,5,6,7,8-hexahydro-1,2,6-triazacyclopenta[b]naphthalene-4a-carboxylic acid methyl ester and pyrrolidine. LCMS: 523 (M+1), retention time 3.86 minutes.

### Example 15

### (R)-1-(4-Fluoro-phenyl)-6-[3-(pyrrolidin-1-yl)-benzenesulfonyl]-1,4,5,6,7,8-hexahydro-1,2,6-triaza-cyclopenta[b]naphthalene-4a-carboxylic acid fluoromethyl ester

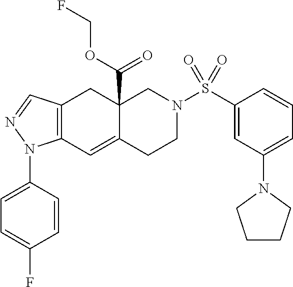 [SMILES: O=C(OCF)[C@]12Cc3cnn(-c4ccc(F)cc4)c3C=C1CCN(S(=O)(=O)c1cccc(N3CCCC3)c1)C2]

The title compound was prepared by the method of Example 13 from (R)-1-(4-fluoro-phenyl)-6-[3-(pyrrolidin-1-yl)-benzenesulfonyl]-1,4,5,6,7,8-hexahydro-1,2,6-triaza-cyclopenta[b]naphthalene-4a-carboxylic acid. LCMS: 555 (M+1), retention time 5.61 minutes. ¹H NMR (400 MHz, CHCl₃-d): δ 7.40-7.35 (m, 3H), 7.29 (t, 1H, J=8.0 Hz), 7.16-7.09 (m, 2H), 6.95 (d, 1H, J=7.3 Hz), 6.83 (t, 1H, J=2.2 Hz), 6.68 (dd, 1H, J=8.3, 2.5 Hz), 6.40 (d, 1H, J=2.3 Hz), 5.79 (dd, 1H, J=50.5, 1.9 Hz), 5.59 (dd, 1H, J=50.4, 1.8 Hz), 4.38 (dd, 1H, J=12.0, 2.0 Hz), 3.88-3.81 (m, 1H), 3.33-3.26 (m, 5H), 2.91-2.80 (m, 1H), 2.63 (d, 1H, J=16.3 Hz), 2.48-2.39 (m, 3H), 2.06-1.98 (m, 4H).

### Example 16

### (R)-1-(4-Fluoro-phenyl)-6-[4-(3,3-difluoropyrrolidin-1-yl)-benzenesulfonyl]-4a-methoxymethyl-4,4a,5,6,7,8-hexahydro-1H-1,2,6-triaza-cyclopenta[b]naphthalene

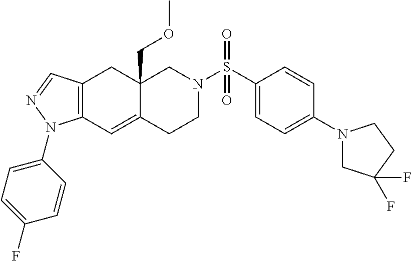 [SMILES: COC[C@]12Cc3cnn(-c4ccc(F)cc4)c3C=C1CCN(S(=O)(=O)c1ccc(N3CCC(F)(F)C3)cc1)C2]

A mixture of BINAP (2,2′-bis(diphenylphosphino)-1,1′-binaphthyl) (23 mg) and tris(dibenzylideneacetone)dipalladium (17 mg) in dry degassed tetrahydrofuran (3 ml) was stirred at room temperature for two minutes. Sodium tert-butoxide (81 mg), 3,3-difluoropyrrolidine hydrochloride (67 mg) and (R)-6-(4-bromo-benzenesulfonyl)-1-(4-fluoro-phenyl)-4a-methoxymethyl-4,4a,5,6,7,8-hexahydro-1H-1,2,6-triaza-cyclopenta[b]naphthalene (100 mg) were added, and the reaction mixture was heated at 65° C. for 3 hours. The resultant mixture was filtered through celite and the solvent was evaporated to provide a red oil. This material was purified by chromatography on silica gel (12 g column, 0 to 50% ethyl acetate in isohexane) to give a yellow solid. Further purification was achieved by preparative HPLC (Waters, Acidic (0.1% Formic acid), Agilent Prep C-18, 5 μm, 21.2×50 mm column, 35-80% MeCN in Water) to provide the title compound as a white solid (53 mg). LCMS: 559 (M+H), retention time 2.64 minutes. ¹H NMR (400 MHz, CHCl₃-d): δ 7.70-7.66 (2H, m), 7.43-7.38 (3H, m), 7.18-7.12 (2H, m), 6.59-6.57 (2H, m), 6.26 (1H, d, J=2.3 Hz), 4.12 (1H, dd, J=11.4, 2.1 Hz), 3.91-3.86 (1H, m), 3.76-3.70 (2H, m), 3.63-3.59 (2H, m), 3.42 (1H, d, J=8.8 Hz), 3.37 (3H, s), 3.13 (1H, d, J=9.0 Hz), 3.08 (1H, d, J=15.7 Hz), 2.77-2.65 (1H, m), 2.59-2.48 (2H, m), 2.38-2.28 (2H, m), 2.17 (1H, d, J=15.4 Hz), 2.02 (1H, d, J=11.0 Hz).

### Example 17

### (R)-1-(4-Fluoro-phenyl)-6-[4-((R)-2-methylpyrrolidin-1-yl)-benzenesulfonyl]-4a-methoxymethyl-4,4a,5,6,7,8-hexahydro-1H-1,2,6-triaza-cyclopenta[b]naphthalene

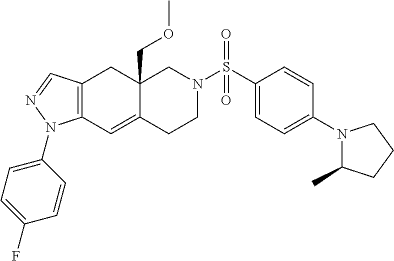 [SMILES: COC[C@]12Cc3cnn(-c4ccc(F)cc4)c3C=C1CCN(S(=O)(=O)c1ccc(N3CCC[C@H]3C)cc1)C2]

The title compound was prepared by the method of Example 16 using (R)-2-methylpyrrolidine and (R)-6-(4-bromo-benzenesulfonyl)-1-(4-fluoro-phenyl)-4a-methoxymethyl-4,4a,5,6,7,8-hexahydro-1H-1,2,6-triaza-cyclopenta[b]naphthalene. LCMS: 537 (M+1), retention time 2.88 minutes. ¹H NMR (400 MHz, CHCl₃-d): δ 7.62-7.58 (2H, m), 7.43-7.38 (3H, m), 7.18-7.12 (2H, m), 6.59-6.56 (2H, m), 6.25 (1H, d, J=2.4 Hz), 5.40 (1H, d, J=51 Hz), 4.11 (1H, dd, J=11.2, 1.8 Hz), 3.99-3.92 (1H, m), 3.91-3.84 (1H, m), 3.47-3.41 (2H, m), 3.37 (3H, s), 3.26-3.19 (1H, m), 3.14 (1H, d, J=9.1 Hz), 3.08 (1H, d, J=15.9 Hz), 2.75-2.66 (1H, m), 2.37-2.29 (2H, m), 2.21-2.02 (5H, m), 1.81-1.73 (1H, m), 1.19 (3H, d, J=6.4 Hz).

### Example 18

### (R)-1-(4-Fluoro-phenyl)-6-[4-((S)-2-(methoxymethyl)pyrrolidin-1-yl)-benzenesulfonyl]-4a-methoxymethyl-4,4a,5,6,7,8-hexahydro-1H-1,2,6-triaza-cyclopenta[b]naphthalene

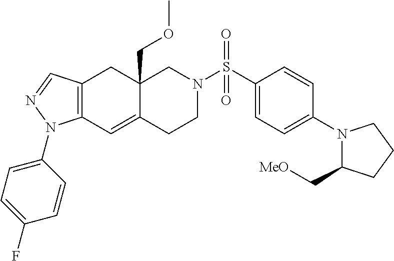 [SMILES: COC[C@@H]1CCCN1c1ccc(S(=O)(=O)N2CCC3=Cc4c(cnn4-c4ccc(F)cc4)C[C@]3(COC)C2)cc1]

The title compound was prepared by the method of Example 16 using (S)-2-(methoxymethyl)pyrrolidine and (R)-6-(4-bromo-benzenesulfonyl)-1-(4-fluoro-phenyl)-4a-methoxymethyl-4,4a,5,6,7,8-hexahydro-1H-1,2,6-triaza-cyclopenta[b]naphthalene. LCMS: 567 (M+1), retention time 2.80 minutes. ¹H NMR (400 MHz, CHCl₃-d): δ 7.63-7.60 (2H, m), 7.43-7.38 (3H, m), 7.18-7.12 (2H, m), 6.67-6.64 (2H, m), 6.25 (1H, d, J=2.3 Hz), 4.11 (1H, dd, J=11.5, 2.1 Hz), 3.98-3.93 (1H, m), 3.90-3.86 (1H, m), 3.49-3.42 (3H, m), 3.38 (6H, s), 3.26-3.19 (2H, m), 3.14 (1H, d, J=9.0 Hz), 3.01 (1H, d, J=16.0 Hz), 2.76-2.67 (1H, m), 2.37-2.29 (2H, m), 2.19 (1H, d, J=16.0 Hz), 2.11-1.97 (5H, m).

### Example 19

### (R)-1-(4-Fluoro-phenyl)-6-[4-((S)-3-methoxypyrrolidin-1-yl)-benzenesulfonyl]-4a-methoxymethyl-4,4a,5,6,7,8-hexahydro-1H-1,2,6-triaza-cyclopenta[b]naphthalene

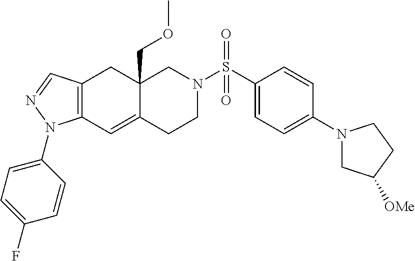 [SMILES: COC[C@]12Cc3cnn(-c4ccc(F)cc4)c3C=C1CCN(S(=O)(=O)c1ccc(N3CC[C@H](OC)C3)cc1)C2]

The title compound was prepared by the method of Example 16 using (S)-3-methoxypyrrolidine hydrochloride and (R)-6-(4-bromo-benzenesulfonyl)-1-(4-fluoro-phenyl)-4a-methoxymethyl-4,4a,5,6,7,8-hexahydro-1H-1,2,6-triaza-cyclopenta[b]naphthalene. LCMS: 553 (M+1), retention time 2.62 minutes. ¹H NMR (400 MHz, CHCl₃-d): δ 7.63-7.60 (2H, m), 7.43-7.39 (3H, m), 7.18-7.12 (2H, m), 6.58-6.55 (2H, m), 6.25 (1H, d, J=2.3 Hz), 4.14-4.09 (2H, m), 3.89-3.85 (1H, m), 3.52-3.41 (5H, m), 3.38 (3H, s), 3.37 (3H, s), 3.13 (1H, d, J=9.2 Hz), 3.08 (1H, d, J=16.2 Hz), 2.76-2.64 (1H, m), 2.37-2.06 (5H, m), 2.02 (1H, d, J=11.3 Hz).

### Example 20

### (R)-1-(4-Fluoro-phenyl)-6-[4-(piperidin-1-yl)-benzenesulfonyl]-4a-methoxymethyl-4,4a,5,6,7,8-hexahydro-1H-1,2,6-triaza-cyclopenta[b]naphthalene

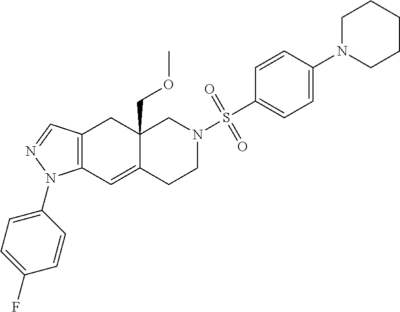 [SMILES: COC[C@]12Cc3cnn(-c4ccc(F)cc4)c3C=C1CCN(S(=O)(=O)c1ccc(N3CCCCC3)cc1)C2]

A solution of (R)-6-(4-fluoro-benzenesulfonyl)-1-(4-fluoro-phenyl)-4a-methoxymethyl-4,4a,5,6,7,8-hexahydro-1H-1,2,6-triaza-cyclopenta[b]naphthalene (97 mg), piperidine (52 mg) and diisopropylethylamine (80 mg) in N-methylpyrrolidine (1 ml) was heated at 100° C. for 9 hours. The crude reaction mixture was purified directly by preparative HPLC (Gilson, Acidic (0.1% Formic acid), Waters X-Select Prep-C18, 5 μm, 19×50 mm column, 5-95% acetonitrile in Water) to provide the title compound as a white solid (24 mg). LCMS: 537 (M+H), retention time 2.95 minutes. ¹H NMR (400 MHz, CHCl₃-d): δ 7.59-7.63 (2H, m), 7.38-7.43 (3H, m), 7.12-7.18 (2H, m), 6.88-6.91 (2H, m), 6.26 (d, 1H, 4.0 Hz), 4.13 (dd, 1H, 12.0, 4.0 Hz), 3.86-3.90 (1H, m), 3.42 (1H, d, J=8.0 Hz), 3.37 (3H, s), 3.32-3.35 (4H, m), 3.13 (1H, d, J=8.0 Hz), 3.08 (1H, d=16.0 Hz), 2.66-2.74 (1H, m), 2.31-2.37 (2H, m), 2.18 (1H, d, J=16.0 Hz), 2.06 (1H, d, J=8.0 Hz), 2.16-2.75 (6H, m).

### Preparation 20a. (R)-6-(4-Fluoro-benzenesulfonyl)-1-(4-fluoro-phenyl)-4a-methoxymethyl-4,4a,5,6,7,8-hexahydro-1H-1,2,6-triaza-cyclopenta[b]naphthalene

The title compound was prepared by the method of Preparation 1c from (R)-1-(4-fluoro-phenyl)-4a-methoxymethyl-1,4,4a,5,7,8-hexahydro-1,2,6-triaza-cyclopenta[b]naphthalene-6-carboxylic acid tert-butyl ester and 4-fluorobenzenesulfonyl chloride. LCMS: 472 (M+1), retention time 2.55 minutes.

### Example 21

### (R)-1-(4-Fluoro-phenyl)-6-[4-(3,3-dimethylpyrrolidin-1-yl)-benzenesulfonyl]-4a-methoxymethyl-4,4a,5,6,7,8-hexahydro-1H-1,2,6-triaza-cyclopenta[b]naphthalene

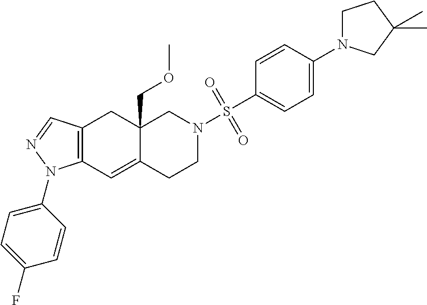 [SMILES: COC[C@]12Cc3cnn(-c4ccc(F)cc4)c3C=C1CCN(S(=O)(=O)c1ccc(N3CCC(C)(C)C3)cc1)C2]

The title compound was prepared by the method of Example 20 using 3,3-dimethylpyrrolidine and (R)-6-(4-fluoro-benzenesulfonyl)-1-(4-fluoro-phenyl)-4a-methoxymethyl-4,4a,5,6,7,8-hexahydro-1H-1,2,6-triaza-cyclopenta[b]naphthalene. LCMS: 551 (M+1), retention time 3.03 minutes. ¹H NMR (400 MHz, CHCl₃-d): δ 7.60 (2H, d, J=8.0 Hz), 7.39-7.42 (3H, m), 7.12-7.16 (2H, m), 7.49-7.53 (2H, d, J=16.0 Hz), 6.25 (1H, d, J=4.0 Hz), 4.11 (1H, dd, J=11.0, 2.0 Hz), 3.85-3.90 (1H, m), 3.40-3.44 (3H, m), 3.37 (3H, s), 3.14 (1H, d, J=9.0 Hz), 3.06-3.10 (3H, m), 2.66-2.75 (1H, m), 2.28-2.36 (2H, m), 2.18 (1H, d, J=16.0 Hz), 2.05 (1H, d, J=11.0 Hz), 1.82 (2H, t, J=7.0 Hz), 1.15 (6H, s).

### Example 22

### Glucocorticoid Receptor Binding Assay

The following is a description of an assay for determining the inhibition of dexamethasone binding of the Human Recombinant Glucocorticoid Receptor:

Binding protocol: Compounds were tested in a binding displacement assay using human recombinant glucocorticoid receptor with ³H-dexamethasone as the ligand. The source of the receptor was recombinant baculovirus-infected insect cells. This GR was a full-length steroid hormone receptor likely to be associated with heat-shock and other endogenous proteins.

The assay was carried out in v-bottomed 96-well polypropylene plates in a final volume of 100 μl containing 0.5 nM GR solution, 2.5 nM 3H-dexamethasone (Perkin Elmer NET119200) in presence of test compounds, test compound vehicle (for total binding) or excess dexamethasone (20 μM, to determine non-specific binding) in an appropriate volume of assay buffer.

For the IC₅₀ determinations, test compounds were tested at 6 concentrations in duplicate. Test compounds were diluted from 10 mM stock in 100% DMSO. The tested solutions were prepared at 2× final assay concentration in 2% DMSO/assay buffer.

All reagents and the assay plate were kept on ice during the addition of reagents. The reagents were added to wells of a v-bottomed polypropylene plate in the following order: 25 μl of 10 nM 3H-dexamethasone solution, 50 μl of TB/NSB/compound solution and 25 μl of 2 nM GR solution. After the additions, the incubation mixture was mixed and incubated for 2.5 hrs at 4° C.

After 2.5 hrs incubation, unbound counts were removed with dextran coated charcoal (DCC) as follows: 15 μl of DCC solution (10% DCC in assay buffer) was added to all wells and mixed (total volume 115 μl). The plate was centrifuged at 4000 rpm for 10 minutes at 4° C. 75 μl of the supernatants was carefully pipetted into an optiplate. 150 μl of scintillation cocktail were added (Microscint-40, Perkin Elmer). The plate was vigorously shaken for approximately 10 minutes and counted on a Topcount.

For the IC₅₀ determinations, the results were calculated as % inhibition [³H]-dexamethasone bound and fitted to sigmoidal curves (fixed to 100 and 0) to obtain IC₅₀ values (concentration of compound that displaces 50% of the bound counts). The IC₅₀ values were converted to Kᵢ (the inhibition constant) using the Cheng-Prusoff equation. Test results are presented in Table 1.

Reagents: Assay buffer: 10 mM potassium phosphate buffer pH 7.6 containing 5 mM DTT, 10 mM sodium molybdate, 100 μM EDTA and 0.1% BSA.

### Example 23

### Human Glucocorticoid Receptor (GR) Fluorescence Polarisation (FP) Binding Assay

The following is a description of a FP assay for measuring compound inhibition of labelled glucocorticoid binding to the human recombinant GR.

The binding affinity of test compounds was determined using a FP binding assay using human recombinant GR (PanVera P2812) and a fluorescent labelled glucocorticoid ligand (Fluorome GS Red) (PanVera P2894). The presence of inhibitors prevents the formation of a GS Red/GR complex resulting in a decrease in the measured polarisation value. The change in polarisation value in the presence of test compounds is used to calculate the binding affinity of the compound for GR.

This assay was performed in 384 well, black, round-bottom, polypropylene micro titre plates in a final volume of 20 μl. The assay contained 5 μl 1 nM GR (final concentration), 5 μl 0.5 nM Fluorome GS Red (final concentration) in the presence of 10 μl test compounds. Positive control wells (high polarisation) receive, 10 μl 2% (v:v) DMSO vehicle (1% (v/v) final concentration)+5 μl 1 nM GR and 5 μl 0.5 nM Fluorome GS Red. Negative control wells (low polarisation) receive 10 μl 2 μM dexamethasone (1 μM final concentration)+5 μl 1 nM GR and 5 μl 0.5 nM Fluorome GS Red. Assay blank background wells (used for normalisation) receive 15 μl 1×GS screening buffer+5 μl GR.

For the IC₅₀ determination (concentration of compound that displaces 50% of the bound GS Red), compounds were tested at eight different concentrations in duplicate in two independently performed experiments. Compounds were prepared as solubilised solids at 10 mM in DMSO. On the day of assay, an 8 point half-log serial dilution (55 μl DMSO+25 μl compound solution) was prepared. A 1:50 dilution (1 μl compound solution+49 μl 1×GR screening buffer) was prepared for each compound. The compounds were prepared at 2× final assay concentration.

The reagents were added to the 384 well micro titre plates in the following order: 10 μl test compound/vehicle/1 μM dexamethasone, 5 μl Fluorome GS Red and 5 μl GR. The plates were mixed and incubated for 4 hour at room temperature. FP was measured using an Envision Excite plate reader with 535 nm excitation and 590 nm emission interference filters.

Milli-polarisation (mP) values were calculated using the below equation—

mP=1000*(S−G*P)/(S+G*P)

where S and P are assay blank background subtracted fluorescence units, G=G-factor (1.07).

Compound IC₅₀ values were calculated by plotting a [compound] v. % inhibition curve and fitting the data to a 4-parameter logistic fit equation. Compound Kᵢ (equilibrium dissociation constant) values were determined from the experimental IC₅₀ values using a ligand depletion correction equation (see below) assuming the antagonists were competitive inhibitors with respect to dexamethasone (Pharmacologic Analysis of Drug Receptor Interactions, 2^{nd} Ed., p 385-410, 1993, Raven Press, New York).

${K}_{i} = \frac{( {L}_{b} ) * ( {IC}_{50} ) * ( {K}_{d} )}{( {L}_{o} ) * ( {R}_{o} ) + {L}_{b} * ( {R}_{o} - {L}_{o} + {L}_{b} - {K}_{d} )}$

| | |
|---|---|
| Equilibrium dissociation constant of GS red ligand (K_{d}) | 0.3 nM |
| Bound tracer concentration (L_{b}) | 0.3 nM |
| Total tracer concentration (Lₒ) | 0.5 nM |
| Total receptor concentration (Rₒ) | 1.0 nM |

Reagents. 10×GR screening buffer (100 mM potassium phosphate pH 7.4, 200 mM Na₂MoO₄, 1 mM EDTA, 20% (v/v) DMSO). To prepare 1×GR screening buffer, combine 1 ml 10×GR screening buffer (PanVera P2814)+1 ml stabilising peptide (PanVera P2815)+7.95 ml 4° C. MQ water. Add 50 μl 1M DTT, vortex and place on ice until use.

### Example 24

### GR Reporter Gene Using SW1353/MMTV-5 Cells

SW1353/MMTV-5 is an adherent human chondrosarcoma cell line that contains endogenous glucocorticoid receptors. It was transfected with a plasmid (pMAMneo-Luc) encoding firefly luciferase located behind a glucocorticoid-responsive element (GRE) derived from a viral promoter (long terminal repeat of mouse mammary tumor virus). A stable cell line SW1353/MMTV-5 was selected with geneticin, which was required to maintain this plasmid. This cell line was thus sensitive to glucocorticoids (dexamethasone) leading to expression of luciferase (EC₅₀^{dex} 10 nM). This dexamethasone-induced response was gradually lost over time, and a new culture from an earlier passage was started (from a cryo-stored aliquot) every three months.

In order to test for a GR-antagonist, SW1353/MMTV-5 cells were incubated with several dilutions of the compounds in the presence of 5×EC₅₀^{dex} (50 nM), and the inhibition of induced luciferase expression was measured using luminescence detected on a Topcount (Britelite Plus kit, Perking Elmer). For each assay, a dose-response curve for dexamethasone was prepared in order to determine the EC₅₀^{dex} required for calculating the Kᵢ from the IC₅₀'s of each tested compound.

SW1353/MMTV-5 cells were distributed in 96-well plates and incubated in medium (without geneticin) for 24 hrs. Dilutions of the compounds in medium+50 nM dexamethasone were added and the plates further incubated for another 24 hrs after which the luciferase expression is measured.

### Example 25

### GR Functional Assay Measuring TAT Induction in Human HepG2 Cells

Glucocorticoid mediated activation of TAT occurs by transactivation of glucocorticoid response elements in the TAT promoter by glucocorticoid receptor-agonist complex. The following protocol describes an assay for measuring induction of TAT by dexamethasone in HepG2 cells (a human liver hepatocellular carcinoma cell line; ECACC, UK).

TAT activity was measured as outlined in the literature by A. Ali et al., J. Med. Chem., 2004, 47, 2441-2452. Dexamethasone induced TAT production with an average EC₅₀ value (half-maximal effect) of 20 nM.

HepG2 cells were cultured using MEME media supplemented with 10% (v/v) foetal bovine serum; 2 mM L-glutamine and 1% (v/v) NEAA at 37° C., 5%/95% (v/v) CO₂/air. The HepG2 cells were counted and adjusted to yield a density of 0.125×10⁶ cells/ml in RPMI 1640 without phenol red, 10% (v/v) charcoal stripped FBS, 2 mM L-glutamine and seeded at 25,000 cells/well in 200 μl into 96 well, sterile, tissue culture micro titre plates, and incubated at 37° C., 5% CO₂ for 24 hours

Growth media was removed and replaced with assay media {RPMI 1640 without phenol red, 2 mM L-glutamine+10 μM forskolin}. Test compounds were screened against a challenge of 100 nM dexamethasone. Compounds were serially half log diluted in 100% (v/v) dimethylsupfoxide from a 10 mM stock. Then an 8-point half-log dilution curve was generated followed by a 1:100 dilution into assay media to give a 10× final assay [compound]: this resulted in final assay [compound] that ranged 10 to 0.003 μM in 0.1% (v/v) dimethylsulfoxide.

Test compounds were pre-incubated with cells in micro-titre plates for 30 minutes at 37° C., 5/95 (v/v) CO₂/air, before the addition of 100 nM dexamethasone and then subsequently for 20 hours to allow optimal TAT induction.

HepG2 cells were then lysed with 30 μl of cell lysis buffer containing a protease inhibitor cocktail for 15 minutes at 4° C. 155 μl of substrate mixture was then added containing 5.4 mM Tyrosine sodium salt, 10.8 mM alpha ketoglutarate and 0.06 mM pyridoxal 5′ phosphate in 0.1M potassium phosphate buffer (pH 7.4). After 2 hours incubation at 37° C. the reaction was terminated by the addition of 15 μl of 10M aqueous potassium hydroxide solution, and the plates incubated for a further 30 minutes at 37° C. The TAT activity product was measured by absorbance at λ 340 nm.

IC₅₀ values were calculated by plotting % inhibition (normalised to 100 nM dexamethasone TAT stimulation) v. [compound] and fitting the data to a 4 parameter logistic equation. IC₅₀ values were converted to Ki (equilibrium dissociation constant) using the Cheng and Prusoff equation, assuming the antagonists were competitive inhibitors with respect to dexamethasone.

TABLE 1
Activity data for selected compounds

| | | GR | GR reporter | GR |
|---|---|---|---|---|
| | | binding | gene | functional |
| Example No. | Structure | Ki nM | Ki nM | Ki nM |
| Example 9 | 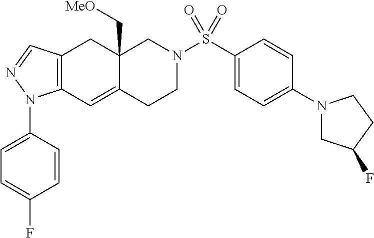 [SMILES: COC[C@]12Cc3cnn(-c4ccc(F)cc4)c3C=C1CCN(S(=O)(=O)c1ccc(N3CC[C@@H](F)C3)cc1)C2] | ++ | ++ | |
| Example 10 | 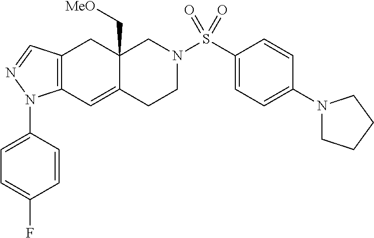 [SMILES: COC[C@]12Cc3cnn(-c4ccc(F)cc4)c3C=C1CCN(S(=O)(=O)c1ccc(N3CCCC3)cc1)C2] | ++ | + | |
| Example 7 | 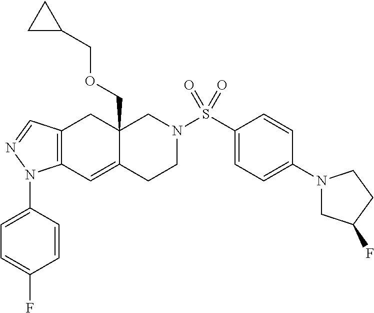 [SMILES: O=S(=O)(c1ccc(N2CC[C@@H](F)C2)cc1)N1CCC2=Cc3c(cnn3-c3ccc(F)cc3)C[C@]2(COCC2CC2)C1] | +++ | +++ | |
| Example 8 | 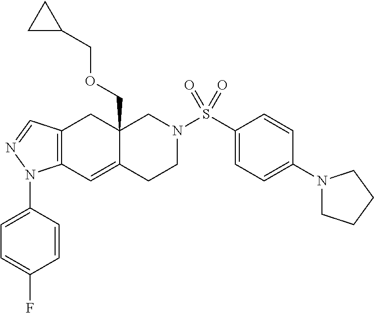 [SMILES: O=S(=O)(c1ccc(N2CCCC2)cc1)N1CCC2=Cc3c(cnn3-c3ccc(F)cc3)C[C@]2(COCC2CC2)C1] | + | +++ | |
| Example 4 | 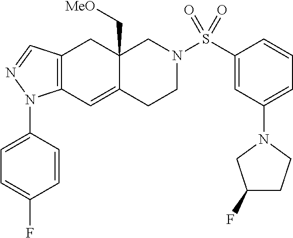 [SMILES: COC[C@]12Cc3cnn(-c4ccc(F)cc4)c3C=C1CCN(S(=O)(=O)c1cccc(N3CC[C@@H](F)C3)c1)C2] | + | ++ | |
| Example 5 | 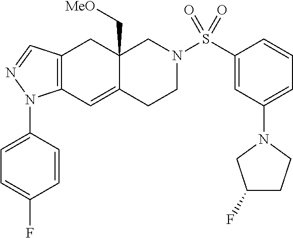 [SMILES: COC[C@]12Cc3cnn(-c4ccc(F)cc4)c3C=C1CCN(S(=O)(=O)c1cccc(N3CC[C@H](F)C3)c1)C2] | ++ | +++ | |
| Example 6 | 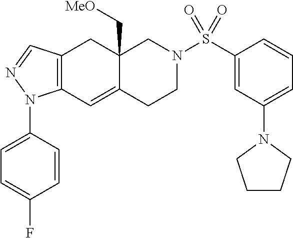 [SMILES: COC[C@]12Cc3cnn(-c4ccc(F)cc4)c3C=C1CCN(S(=O)(=O)c1cccc(N3CCCC3)c1)C2] | ++ | ++ | |
| Example 1 | 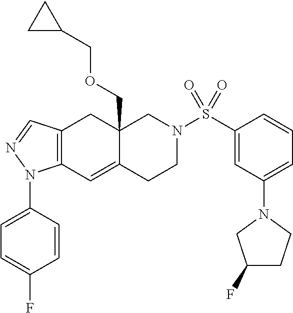 [SMILES: O=S(=O)(c1cccc(N2CC[C@@H](F)C2)c1)N1CCC2=Cc3c(cnn3-c3ccc(F)cc3)C[C@]2(COCC2CC2)C1] | + | +++ | |
| Example 2 | 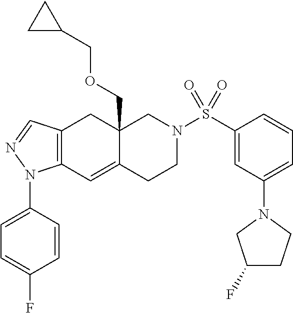 [SMILES: O=S(=O)(c1cccc(N2CC[C@H](F)C2)c1)N1CCC2=Cc3c(cnn3-c3ccc(F)cc3)C[C@]2(COCC2CC2)C1] | ++ | +++ | |
| Example 3 | 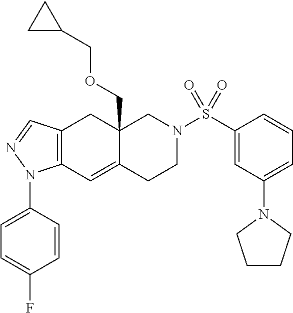 [SMILES: O=S(=O)(c1cccc(N2CCCC2)c1)N1CCC2=Cc3c(cnn3-c3ccc(F)cc3)C[C@]2(COCC2CC2)C1] | + | +++ | |
| Example 11 | 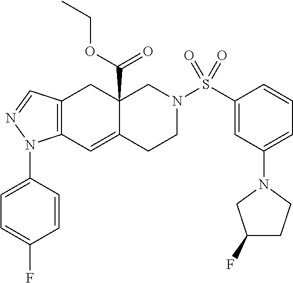 [SMILES: CCOC(=O)[C@]12Cc3cnn(-c4ccc(F)cc4)c3C=C1CCN(S(=O)(=O)c1cccc(N3CC[C@@H](F)C3)c1)C2] | ++ | ++ | |
| Example 12 | 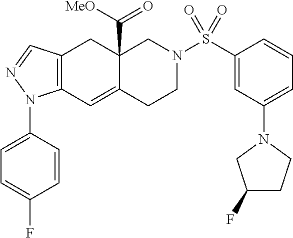 [SMILES: COC(=O)[C@]12Cc3cnn(-c4ccc(F)cc4)c3C=C1CCN(S(=O)(=O)c1cccc(N3CC[C@@H](F)C3)c1)C2] | + | + | |
| Example 13 | 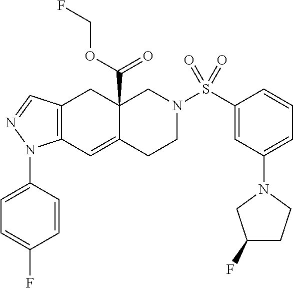 [SMILES: O=C(OCF)[C@]12Cc3cnn(-c4ccc(F)cc4)c3C=C1CCN(S(=O)(=O)c1cccc(N3CC[C@@H](F)C3)c1)C2] | ++ | + | |
| Example 15 | 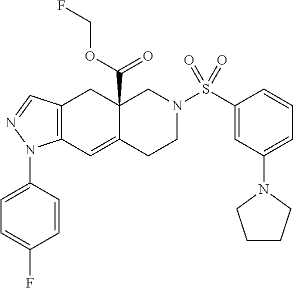 [SMILES: O=C(OCF)[C@]12Cc3cnn(-c4ccc(F)cc4)c3C=C1CCN(S(=O)(=O)c1cccc(N3CCCC3)c1)C2] | ++ | + | |
| Example 14 | 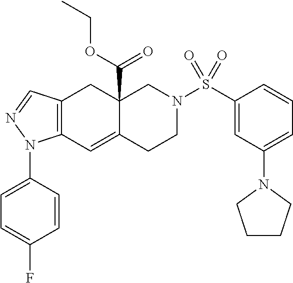 [SMILES: CCOC(=O)[C@]12Cc3cnn(-c4ccc(F)cc4)c3C=C1CCN(S(=O)(=O)c1cccc(N3CCCC3)c1)C2] | ++ | ++ | |
| Example 16 | 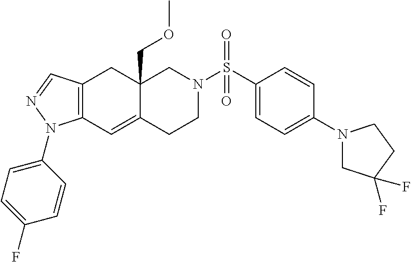 [SMILES: COC[C@]12Cc3cnn(-c4ccc(F)cc4)c3C=C1CCN(S(=O)(=O)c1ccc(N3CCC(F)(F)C3)cc1)C2] | +++ | | + |
| Example 17 | 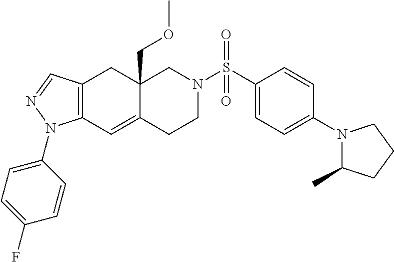 [SMILES: COC[C@]12Cc3cnn(-c4ccc(F)cc4)c3C=C1CCN(S(=O)(=O)c1ccc(N3CCC[C@H]3C)cc1)C2] | +++ | | + |
| Example 18 | 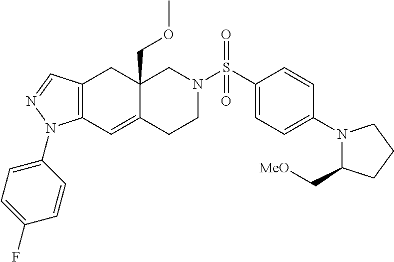 [SMILES: COC[C@@H]1CCCN1c1ccc(S(=O)(=O)N2CCC3=Cc4c(cnn4-c4ccc(F)cc4)C[C@]3(COC)C2)cc1] | ++ | | ++ |
| Example 19 | 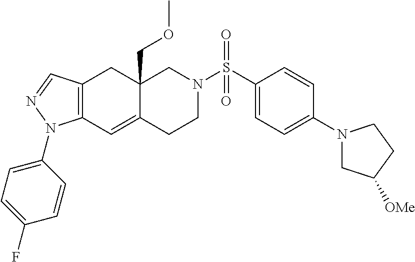 [SMILES: COC[C@]12Cc3cnn(-c4ccc(F)cc4)c3C=C1CCN(S(=O)(=O)c1ccc(N3CC[C@H](OC)C3)cc1)C2] | +++ | | + |
| Example 20 | 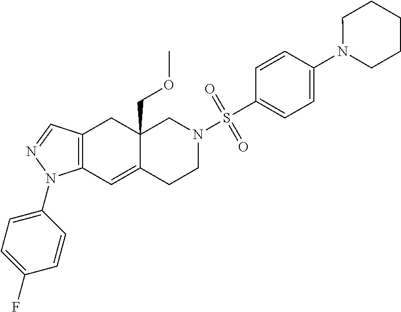 [SMILES: COC[C@]12Cc3cnn(-c4ccc(F)cc4)c3C=C1CCN(S(=O)(=O)c1ccc(N3CCCCC3)cc1)C2] | +++ | | ++ |
| Example 21 | 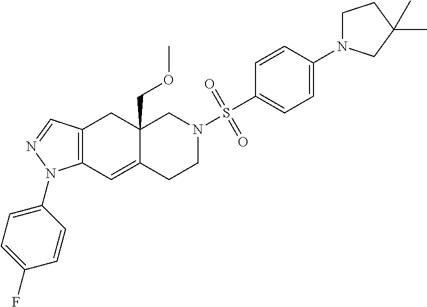 [SMILES: COC[C@]12Cc3cnn(-c4ccc(F)cc4)c3C=C1CCN(S(=O)(=O)c1ccc(N3CCC(C)(C)C3)cc1)C2] | +++ | | + |

In Table 1, Examples 1-15 were tested in the GR binding assay described in Example 22, and compounds of examples 16-21 were tested in the GR binding assay described in Example 23. GR binding activity with a Kᵢ value of less than 0.5 nM are designated with +++; compounds with a Kᵢ value from 0.5 nM to less than 1.0 nM are designated with ++; and compounds with a Kᵢ value of at least 1.0 nM are designated with +. GR reporter gene activity with a Kᵢ value of less than 10 nM are designated with +++, compounds with a Kᵢ value from 10 nM to less than 20 nM are designated with ++; and compounds with a Kᵢ value of at least 20 nM are designated with +. GR functional activity with a Kᵢ value of less than 50 nM are designated with +++, compounds with a Kᵢ value of 50 nM to 100 nM are designated with ++; and compounds with a Kᵢ value greater than 100 nM are designated with +.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A compound having the formula: 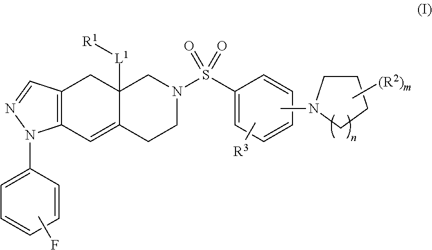
wherein
L¹ is selected from the group consisting of C₁₋₆ alkylene and —C(O)—;
R¹ is —OR^{1a};
each R^{1a} is independently selected from the group consisting of C₁₋₆ alkyl, C₁₋₆ haloalkyl, C₃₋₈ cycloalkyl, and C₁₋₆ alkyl-C₃₋₈ cycloalkyl;
R² is selected from the group consisting of hydrogen, halogen, C₁₋₆ alkyl, C₁₋₆ alkoxy, C₁₋₆ alkyl-C₁₋₆ alkoxy, C₁₋₆ haloalkyl, and C₁₋₆ haloalkoxy;
R³ is selected from the group consisting of hydrogen, halogen, C₁₋₆ alkyl, and C₁₋₆ haloalkyl;
each of subscripts m and n are independently 1 or 2;
or salts and isomers thereof.

2. The compound of claim 1, wherein the group L¹-R¹ is selected from the group consisting of —CH₂OR^{1a} and —C(O)OR^{1a}.

3. The compound of claim 1, wherein R^{1a} is selected from the group consisting of C₁₋₆ alkyl, C₁₋₆ haloalkyl, and C₁₋₆alkylC₃₋₈ cycloalkyl.

4. The compound of claim 1, wherein the group L¹-R¹ is selected from the group consisting of methoxymethyl, ethoxymethyl, isopropoxymethyl, (fluoromethoxy)methyl, (difluoromethoxy)methyl, (trifluoromethoxy)methyl, (cyclopropylmethoxy)methyl, (cyclobutylmethoxy)methyl, methyl carboxylate, ethyl carboxylate, isopropyl carboxylate, fluoromethyl carboxylate, cyclopropyl carboxylate, cyclobutyl carboxylate, cyclopropylmethyl carboxylate, and cyclobutylmethyl carboxylate.

5. The compound of claim 1, wherein the group L¹-R¹ is selected from the group consisting of methoxymethyl, ethoxymethyl, (cyclopropylmethoxy)methyl, methyl carboxylate, ethyl carboxylate and fluoromethyl carboxylate.

6. The compound of claim 1, wherein
R² is selected from the group consisting of hydrogen, halogen, and C₁₋₆ alkoxy; and
subscript m is 1.

7. The compound of claim 1, wherein R² is selected from the group consisting of H and F.

8. The compound of claim 1, wherein
R^{1a} is selected from the group consisting of C₁₋₆ alkyl, C₁₋₆ haloalkyl, and C₁₋₆ alkyl-C₃₋₈ cycloalkyl;
R² is selected from the group consisting of hydrogen, halogen, and C₁₋₆ alkoxy;
R³ is H; and
each of subscripts m and n are 1.

9. The compound of claim 1, having the structure:

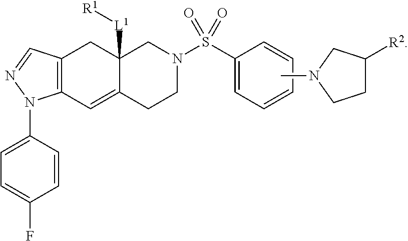

10. The compound of claim 1, wherein
the group L¹-R¹ is selected from the group consisting of methoxymethyl, ethoxymethyl, (cyclopropylmethoxy)methyl, methyl carboxylate, ethyl carboxylate and fluoromethyl carboxylate;
R² is selected from the group consisting of H and F;
R³ is H; and
each of subscripts m and n are 1.

11. The compound of claim 1, having the structure selected from the group consisting of:

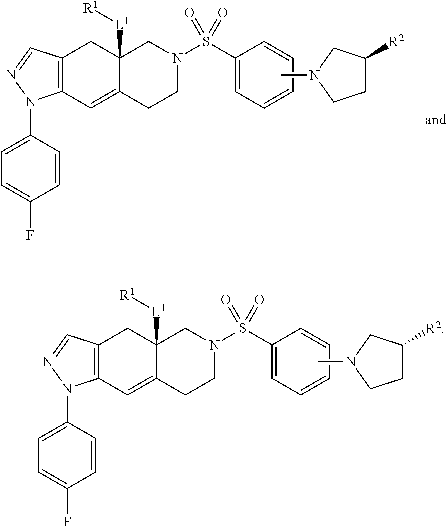

12. The compound of claim 1, having the structure selected from the group consisting of:

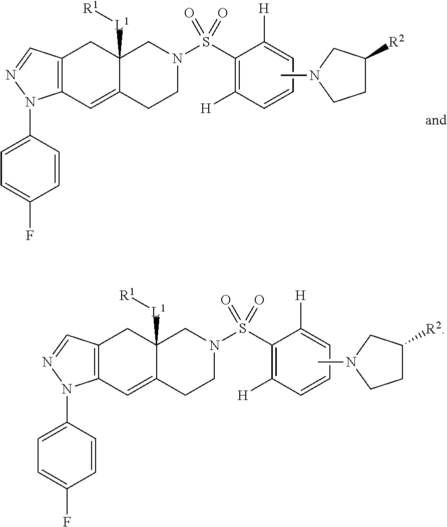

13. The compound of claim 1, selected from the group consisting of:
(R)-4a-Cyclopropylmethoxymethyl-1-(4-fluorophenyl)-6-[3-((R)-3-fluoropyrrolidin-1-yl)-benzenesulfonyl]-4,4a,5,6,7,8-hexahydro-1H-1,2,6-triaza-cyclopenta[b]naphthalene;
(R)-4a-Cyclopropylmethoxymethyl-1-(4-fluorophenyl)-6-[3-((S)-3-fluoropyrrolidin-1-yl)-phenyl-3-sulfonyl]-4,4a,5,6,7,8-hexahydro-1H-1,2,6-triaza-cyclopenta[b]naphthalene;
(R)-4a-Cyclopropylmethoxymethyl-1-(4-fluorophenyl)-6-[3-(pyrrolidin-1-yl)-benzenesulfonyl]-4,4a,5,6,7,8-hexahydro-1H-1,2,6-triaza-cyclopenta[b]naphthalene;
(R)-1-(4-Fluorophenyl)-6-[3-((R)-3-fluoropyrrolidin-1-yl)-benzenesulfonyl]-4a-methoxymethyl-4,4a,5,6,7,8-hexahydro-1H-1,2,6-triaza-cyclopenta[b]naphthalene;
(R)-1-(4-Fluorophenyl)-6-[3-((S)-3-fluoropyrrolidin-1-yl)-benzenesulfonyl]-4a-methoxymethyl-4,4a,5,6,7,8-hexahydro-1H-1,2,6-triaza-cyclopenta[b]naphthalene;
(R)-1-(4-Fluoro-phenyl)-4a-methoxymethyl-6-[3-(-pyrrolidin-1-yl)-benzenesulfonyl)-4,4a,5,6,7,8-hexahydro-1H-1,2,6-triaza-cyclopenta[b]naphthalene;
(R)-4a-Cyclopropylmethoxymethyl-1-(4-fluoro-phenyl)-6-[4-((R)-3-fluoro-pyrrolidin-1-yl)-benzenesulfonyl]-4,4a,5,6,7,8-hexahydro-1H-1,2,6-triaza-cyclopenta[b]naphthalene;
(R)-4a-Cyclopropylmethoxymethyl-1-(4-fluoro-phenyl)-6-[4-(pyrrolidin-1-yl)-benzenesulfonyl]-4,4a,5,6,7,8-hexahydro-1H-1,2,6-triaza-cyclopenta[b]naphthalene;
(R)-1-(4-Fluoro-phenyl)-6-[4-((R)-3-fluoro-pyrrolidin-1-yl)-benzenesulfonyl]-4a-methoxymethyl-4,4a,5,6,7,8-hexahydro-1H-1,2,6-triaza-cyclopenta[b]naphthalene;
(R)-1-(4-Fluoro-phenyl)-6-[4-(pyrrolidin-1-yl)-benzenesulfonyl]-4a-methoxymethyl-4,4a,5,6,7,8-hexahydro-1H-1,2,6-triaza-cyclopenta[b]naphthalene;
(R)-1-(4-Fluoro-phenyl)-6-[3-((R)-3-fluoro-pyrrolidin-1-yl)-benzenesulfonyl]-1,4,5,6,7,8-hexahydro-1,2,6-triaza-cyclopenta[b]naphthalene-4a-carboxylic acid ethyl ester;
(R)-1-(4-Fluoro-phenyl)-6-[3-((R)-3-fluoro-pyrrolidin-1-yl)-benzenesulfonyl]-1,4,5,6,7,8-hexahydro-1,2,6-triaza-cyclopenta[b]naphthalene-4a-carboxylic acid methyl ester;
(R)-1-(4-Fluoro-phenyl)-6-[3-((R)-3-fluoro-pyrrolidin-1-yl)-benzenesulfonyl]-1,4,5,6,7,8-hexahydro-1,2,6-triaza-cyclopenta[b]naphthalene-4a-carboxylic acid fluoromethyl ester;
(R)-1-(4-Fluoro-phenyl)-6-[3-(pyrrolidin-1-yl)-benzenesulfonyl]-1,4,5,6,7,8-hexahydro-1,2,6-triaza-cyclopenta[b]naphthalene-4a-carboxylic acid ethyl ester;
(R)-1-(4-Fluoro-phenyl)-6-[3-(pyrrolidin-1-yl)-benzenesulfonyl]-1,4,5,6,7,8-hexahydro-1,2,6-triaza-cyclopenta[b]naphthalene-4a-carboxylic acid fluoromethyl ester,
(R)-1-(4-Fluoro-phenyl)-6-[4-(3,3-difluoropyrrolidin-1-yl)-benzenesulfonyl]-4a-methoxymethyl-4,4a,5,6,7,8-hexahydro-1H-1,2,6-triaza-cyclopenta[b]naphthalene,
(R)-1-(4-Fluoro-phenyl)-6-[4-((R)-2-methylpyrrolidin-1-yl)-benzenesulfonyl]-4a-methoxymethyl-4,4a,5,6,7,8-hexahydro-1H-1,2,6-triaza-cyclopenta[b]naphthalene,
(R)-1-(4-Fluoro-phenyl)-6-[4-((S)-2-(methoxymethyl)pyrrolidin-1-yl)-benzenesulfonyl]-4a-methoxymethyl-4,4a,5,6,7,8-hexahydro-1H-1,2,6-triaza-cyclopenta[b]naphthalene,
(R)-1-(4-Fluoro-phenyl)-6-[4-((S)-3-methoxypyrrolidin-1-yl)-benzenesulfonyl]-4a-methoxymethyl-4,4a,5,6,7,8-hexahydro-1H-1,2,6-triaza-cyclopenta[b]naphthalene,
(R)-1-(4-Fluoro-phenyl)-6-[4-(piperidin-1-yl)-benzenesulfonyl]-4a-methoxymethyl-4,4a,5,6,7,8-hexahydro-1H-1,2,6-triaza-cyclopenta[b]naphthalene, and
(R)-1-(4-Fluoro-phenyl)-6-[4-(3,3-dimethylpyrrolidin-1-yl)-benzenesulfonyl]-4a-methoxymethyl-4,4a,5,6,7,8-hexahydro-1H-1,2,6-triaza-cyclopenta[b]naphthalene.

14. A pharmaceutical composition comprising a compound of claim 1 and a pharmaceutically acceptable excipient.

15. A method of modulating a glucocorticoid receptor, comprising contacting a glucocorticoid receptor with a compound of claim 1, thereby modulating the glucocorticoid receptor.

16. A method of treating a disorder through antagonizing a glucocorticoid receptor, the method comprising administering to a subject in need thereof, a therapeutically effective amount of a compound of claim 1, thereby treating the disorder.
